(19) United States
(12) Patent Application Publication
(10) Pub. No.: US 2014/0371427 A1
(43) Pub. Date: Dec. 18, 2014

(54) IgG2 DISULFIDE ISOFORM SEPARATION

(71) Applicant: AMGEN INC., Thousand Oaks, CA (US)

(72) Inventors: Thomas M. Dillon, Ventura, CA (US); Yi-Te Chou, Simi Valley, CA (US); Yautyan Chen, Thousand Oaks, CA (US); Pavel Bondarenko, Thousand Oaks, CA (US); Jed J. Wiltzius, Pacifica, CA (US); Diana Woehle, Simi Valley, CA (US)

(73) Assignee: AMGEN INC., Thousand Oaks, CA (US)

(21) Appl. No.: 14/363,735

(22) PCT Filed: Dec. 7, 2012
(86) PCT No.: PCT/US12/68614

§ 371 Date: Jun. 6, 2014

Related U.S. Application Data
(60) Provisional application No. 61/568,018, filed on Dec. 7, 2011.

(51) Int. Cl. C07K 1/18

(52) U.S. Cl. CPC C07K 1/18
USPC 530/387.1

(57) ABSTRACT

Methods for producing an IgG2 antibody preparation enriched for one of several IgG2 structural isoforms, differing by disulfide connectivity in the hinge region of the antibody, are disclosed.

### BACKGROUND

Human IgG2 antibodies have been shown to be comprised of three major structural isoforms IgG2-A, -B, and -AB (Wypych et al., 2008; Dillon et al., 2008b). This structural heterogeneity is due to different light chain to heavy chain connectivity in each isoform. These structural isoforms are inherent in recombinant IgG2 monoclonal antibodies (mAbs) as well as naturally occurring IgG2 in the human body. Since the discovery of the IgG2 disulfide isoforms, it has been apparent that the individual isoforms can have unique and different structural and functional properties (Dillon et al., 2008b), including differences in potency or other quality attributes including Fcγ receptor binding, viscosity, stability, and particle formation. Current requirements from regulatory agencies indicate that if the IgG2 disulfide isoforms have different potencies (or other critical attribute), their relative abundances may need to be monitored and controlled (Cherney, 2010). Therefore, if the disulfide isoforms are deemed a critical quality attribute for a therapeutic mAb, process monitoring controls may be required. Reversed phase HPLC analysis was described as one of the methods of monitoring the IgG2 disulfide isoforms (Dillon et al., 2008a). Since differences in quality attributes for IgG2 isoforms can be present, it has become more important that each of the individual isoforms be characterized early in clinical development. Enrichment of the individual IgG2 isoforms is a prerequisite for such characterization. Previously, IgG2 disulfide isoforms were enriched by redox treatment (Dillon et al., 2006b; Dillon et al., 2006b; Dillon et al., 2008b) or weak cation exchange chromatography (Wypych et al., 2008), which have produced modest quantities of moderately pure fractions. However, efficient characterization and manufacture of the isoforms would benefit from a higher degree of purity, and higher production yields. There thus remains a need for separation techniques that are capable of producing fractions of isoforms that are more highly purified than those produced by the methods summarized above.

### BRIEF DESCRIPTION OF THE DRAWINGS

FIGS. 1A and 1B show a comparison of low pH (≦5) CEX separation of mAb1 disulfide isoforms using the standard method (Dionex WCX; FIG. 1A) and the SCX method described herein (FIG. 1B). This comparison was done using analytical size columns (4.6×300 mm for Dionex and 4.6×100 mm for YMC SCX) operated with on an Agilent 1100 HPLC. As shown, the YMC SCX column was able to provide additional resolution of the isoforms relative to the Dionex WCX column.

FIG. 2 shows the percent mAb1 disulfide isoforms (isoform species indicated on legend) in cation exchange fractions (fraction number along x-axis) collected from a 500 mL YMC-SCX column. Fractions were collected after a 1-gram injection of mAb1 material and salt/pH gradient (salt increasing and pH decreasing with increasing fraction number). As shown, IgG2-B content was highest in the first fractions while IgG2-A/B and -A was greatest in later fractions. The elution order from earliest to latest was IgG2-B, IgG-A/B, and IgG2-A. The percentage of the isoforms was determined by the reversed-phase HPLC.

FIGS. 3A, 3B, 3C and 3D show reversed-phase chromatograms of mAb1 CEX fractions separated using the YMC BioPro SP-F SCX column (FIG. 2). mAb1 bulk is shown in FIG. 3A. Earlier eluting CEX fractions (FIGS. 3B & 3C) contained highly enriched peaks-1 & 2, respectively (IgG2-B & IgG2-A/B, respectively), while the later eluting fraction (FIG. 3D) contained highly enriched peaks-3 & 4 (IgG2-A species).

FIG. 4 shows reversed-phase chromatograms of mAb1 bulk (solid line) and Protein L elution fraction (dashed line). mAb1 bulk material was loaded on a 5 mL semi-preparative Pierce chromatography column (PN-89929) and eluted with a 100 mM glycine buffer pH 2.8. The collected fraction was injected onto a reversed-phase column. Significant enrichment of IgG2-AB and IgG2-A species was achieved using the Protein L affinity column method.

FIG. 5 shows fast protein liquid chromatography (FPLC) purification diagram for mAb1 recorded on AKTA system equipped with Protein L column using bind and elute method. mAb1 material was loaded on a 24 mL preparative Protein L column. mAb1 was diluted in PBS pH 7.2 (1:1) and loaded onto the column. The running buffer was PBS pH 7.2 and the elution buffer was 100 mM glycine pH 2.8. The pH is shown by the grey line and corresponds to the pH units along the y-axis. The data show that the majority of mAb1 was not retained by the column and was removed through washing. The retained mAb1 material was eluted at low pH (˜4.2) and collected for analysis.

FIGS. 6A and 6B show reversed-phase chromatograms of mAb1 bulk (solid grey line) and Protein L flow through material (FIG. 6A, dashed line) and elution material (FIG. 6B, dashed line; mAb1 bulk is shown in FIG. 6B as a solid black line). mAb1 bulk material was loaded on a 24 mL Protein L preparative FPLC column and eluted with a 100 mM glycine buffer pH 2.8. The collected fractions were buffer exchanged and injected onto a reversed-phase column. Significant enrichment of IgG2-B and IgG2-A species were achieved using the Protein L affinity column method.

FIGS. 7A, 7B and 7C show reversed-phase chromatograms of Protein L fractions generated by FPLC (AKTA) purification and fractionation of another IgG2 antibody mAb2. mAb2 material was loaded on a 24 mL Protein L preparative column after dilution in PBS pH 7.2 (1:1). The running buffer was PBS pH 7.2 and the elution buffer was 100 mM glycine pH 2.8. Unlike in the case with mAb1, no protein was detected in the flow through, indicating that all mAb2 disulfide isoforms were retained. The eluted fractions were analyzed by reversed-phase HPLC. Bulk material of mAb2 contained B, A/B, A1 and A2 isoforms (FIG. 7A). IgG2-B & A/B were eluted first as the pH was lowered (FIG. 7B). The IgG2-A species (A1 & A2) were eluted when the pH reached ˜3 (FIG. 7C).

FIGS. 8A, 8B and 8C show reversed-phase chromatograms of the Protein L fractions following FPLC (AKTA) purification and fractionation of mAb3. mAb3 material was loaded on a 275 mL Protein L column. The running buffer was 25 mM MOPS pH 6.5 and the elution buffer was 100 mM glycine pH 2.8. No protein was detected in the flow through, showing that all mAb3 disulfide Isoforms were retained. The eluted fractions were analyzed by reversed-phase analysis. Bulk material of mAb2 contained B, A/B, A1 and A2 isoforms (FIG. 8A). IgG2-B & A/B were eluted first as the pH was lowered (FIG. 8B). The IgG2-A species (A1 & A2) were eluted when the pH reached ˜3 (FIG. 8C).

FIGS. 9A, 9B and 9C show reversed-phase chromatograms of Protein L fractions following FPLC (AKTA) purification and fractionation of mAb3, using different buffers from those used for experiments shown in FIGS. 8A, 8B and 8C mAb3 material was loaded on a 275 mL large preparative Protein L column. The running buffers were Gentle Ag/Ab Binding and Elution buffers allowing for near neutral pH elution. No protein was detected in the flow through, showing that all mAb3 disulfide isoforms were retained. The eluted fractions were analyzed by reversed-phase HPLC. Bulk material of mAb3 contained B, A/B, A1 and A2 isoforms (FIG. 9A). IgG2-B & IgG2-A/B were eluted first as the pH was lowered to mildly acidic pH (FIG. 9B). The IgG2-A species (A1 & A2) were eluted when the pH reached ˜3 (FIG. 9C).

FIG. 10 shows size exclusion chromatography binding assay for mAb1-B & mAb1-A enriched fractions and anti-human IgG2 HP-6014 control material. The mAb1-B & mAb1-A material is ˜65% pure relative to each isoform. As shown, the material enriched in the IgG2-A isoform has near complete binding while the IgG2-B enriched material remained mainly unbound.

FIG. 11 shows size exclusion chromatography binding assay for mAb2-B & mAb2-A enriched fractions and anti-human IgG2 HP-6014 control material. The mAb2-B & mAb2-A material is ˜65% pure relative to each isoform. As shown, the material enriched in the IgG2-A isoform has near complete binding while the IgG2-B enriched material remained mainly unbound.

FIG. 12 shows size exclusion chromatography binding assay for mAb7-B & mAb7-A enriched fractions and anti-human IgG2 HP-6014 control material. The mAb7-B & mAb7-A material is ˜65% pure relative to each isoform. As shown, the material enriched in the IgG2-A isoform has near complete binding while the IgG2-B enriched material remained mainly unbound.

FIG. 13 shows size exclusion chromatography binding assay for mAb1-β isoform, mAb1-A isoform and anti-IgG2 HP-6002. The mAb1-B and mAb1-A material is ˜65% pure relative to each isoform. As shown, all isoforms had similar binding to anti-human IgG2 clone HP-6002.

FIG. 14 shows size exclusion chromatography binding assay for mAb7-β isoform, mAb7-A isoform and anti-IgG2 HP-6002. The mAb7-B and mAb7-A material is ˜65% pure relative to each isoform. As shown, all isoforms had similar binding to anti-human IgG2 clone HP-6002.

FIGS. 15A, 15B and 15C shows reversed-phase chromatograms of mAb3 fractions following FPLC (AKTA) purification and fractionation by immobilized anti-Hu IgG2 HP-6014. mAb3 material was loaded on an anti-Hu IgG2 affinity column. The running buffer was PBS pH 7.2 and the elution buffer was 100 mM glycine pH 2.8. The eluted fractions were analyzed by reversed-phase HPLC. Bulk material of mAb2 contained B, A/B, A1 and A2 isoforms (FIG. 15B). The majority of mAb3 was not retained by the column and was eluted in the F/T (FIG. 15A). The retained mAb3 material was eluted at low pH (˜3.8) and collected for analysis. The IgG2-A species (A1 & A2) were eluted when the pH reached ˜3.8 and were the main retained components (FIG. 15C).

FIG. 16 shows a flow chart for enrichment of mAb1 IgG2 disulfide isoforms B, A/B, A1 and A2.

FIG. 17 shows a flow chart for enrichment of mAb7 IgG2 disulfide isoforms B, A/B, and A.

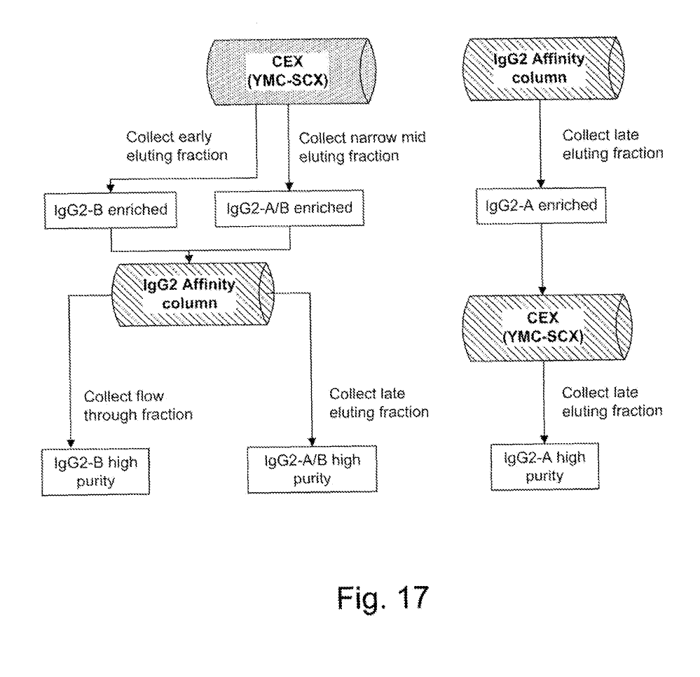

FIG. 18 shows a chromatogram of elution of IgG2 from a 7 cm preparative cation exchange column (12 g/L resin load; detection at 280 nm).

FIG. 19 shows IgG2 disulfide isoform separation by preparative CEX. The solid line corresponds to the concentration of IgG2 in the elution fractions; dashed and dotted lines correspond to the percent peak area of the disulfide isoforms measured in each fraction.

FIG. 20 shows a chromatogram of elution of IgG2 from a 10 cm preparative cation exchange column (2.1 g/L resin load; detection at 280 nm).

FIG. 21 shows IgG2 disulfide isoform separation by preparative CEX. The solid line corresponds to the concentration of IgG2 in the elution fractions; dashed and dotted lines correspond to the percent peak area of the disulfide isoforms measured in each fraction.

### SUMMARY

In one aspect, the invention includes a method of producing an IgG2 antibody preparation enriched for at least one of several IgG2 structural variants which differ by disulfide connectivity in the hinge region, comprising (A) contacting a solution containing a recombinantly-produced IgG2 antibody with a first matrix selected from the group consisting of a strong cation exchange (SCX) matrix, an IgG2 (e.g., HP-6014) affinity matrix and a Protein L matrix, and (B) eluting two or more first elution fractions from the first matrix, wherein (i) the IgG2 antibody solution to be subjected to the method elutes off the first matrix as two or more separate forms corresponding to two or more IgG2 structural variants, and (ii) at least one of the two or more first elution fractions is enriched for at least one of the IgG2 structural variants which differ by disulfide connectivity in the hinge region. In one embodiment, the method further comprises contacting at least one of the two or more first elution fractions with a second matrix selected from the group consisting of an SCX matrix, an IgG2 (e.g., HP-6014) affinity matrix and a Protein L matrix, and eluting two or more second elution fractions off the second matrix, wherein at least one of the two or more second elution fractions is further enriched for at least one of the IgG2 structural variants. In one embodiment, the first matrix is selected from the group consisting of a SCX matrix and an IgG2 (e.g., HP-6014) affinity matrix. In another embodiment, the first matrix is an SCX matrix. In another embodiment, the first matrix is an IgG2 (e.g., HP-6014) affinity matrix. In a more specific embodiment, the first matrix is an SCX matrix and the second matrix is an IgG2 (e.g., HP-6014) affinity matrix. In another more specific embodiment, the first matrix is an SCX matrix and the second matrix is a Protein L matrix. In another more specific embodiment, the first matrix is an IgG2 (e.g., HP-6014) affinity matrix and the second matrix is an SCX matrix.

In any of the above embodiments, the SCX matrix may comprise YMC-SCX. In any of the above embodiments, the eluting from the first or second matrix may be performed using a low pH buffer, e.g., a buffer having a pH of between about 2 and 3, about 3 and 4, about 4 and 5, and about 5 and 6. In specific embodiments, the elution buffer has a pH of less than or equal to 5, about 4.2, about 3.8, or about 2.8. In any of the above aspects and embodiments, a pH step elution or a pH gradient elution may be employed, e.g., a pH gradient from pH˜7.2→˜2.8. In any of the above aspects and embodiments, a salt step elution or a salt gradient elution may be employed, e.g., a gradient elution which increases the salt concentration from ˜100 mM to ˜250 mM, e.g., NaCl. In any of the above aspect or embodiments, the IgG2 antibody preparation may be enriched for at least one of the IgG2 structural variants to a level of at least 20%, at least 30%, at least 40%, or at least 50% purity of a desired IgG2 structural variant.

In any of the above embodiments, the matrixes may be packed in a protein purification column, e.g., an analytical scale column, a semi-preparative scale column, or a preparative scale column. The column may be, for example, may have a volume of 1 ml or more, 2 ml or more, 3 ml or more, 4 ml or more, 5 ml or more, 6 ml or more, 7 ml or more, 8 ml or more, 9 ml or more, 10 ml or more, 15 ml or more, 25 ml or more, 50 ml or more, 100 ml or more, 200 ml or more, 500 ml or more, 1 l or more, 10 l or more, 100 l or more, 1000 l or more. The column may employ resin beads having diameters of 5 microns or greater, 10 microns or greater, 15 microns or greater, 20 microns or greater, 25 microns or greater, 26 microns or greater, 27 microns or greater, 28 microns or greater, 29 microns or greater, 30 microns or greater, 35 microns or greater, or 40 microns or greater. The column diameter may be, e.g., 1 cm or greater, 2 cm or greater, 3 cm or greater, 4 cm or greater, 5 cm or greater, 6 cm or greater, 7 cm or greater, 8 cm or greater, 9 cm or greater, 10 cm or greater, cm or greater, 30 cm or greater, 40 cm or greater, 50 cm or greater, or 100 cm or greater. The column may employ flow rates of, e.g., 10 cm/hr or more, 25 cm/hr or more, 50 cm/hr or more, 100 cm/hr or more, 125 cm/hr or more, 150 cm/hr or more, 175 cm/hr or more, 200 cm/hr or more, 400 cm/hr or more, 600 cm/hr or more, 800 cm/hr or more, or 1000 cm/hr or more. The amount of mAb loaded on the column may be 0.1 g/L or more, 0.5 g/L or more, 1 g/L or more, 2 g/L or more, 4 g/L or more, 5 g/L or more, 6 g/L or more, 7 g/L or more, 8 g/L or more, 9 g/L or more, 10 g/L or more, 11 g/L or more, 12 g/L or more, 13 g/L or more, 14 g/L or more, 15 g/L or more, 20 g/L or more, 25 g/L or more, or 30 g/L or more. The column may employ, e.g., a single gradient of increasing salt for the peak elution, with, e.g., 20 or fewer column volumes (CV), 15 or fewer CV, 14 or fewer CV, 13 or fewer CV, 12 or fewer CV, 11 or fewer CV, 10 or fewer CV, 9 or fewer CV, 8 or fewer CV, 7 or fewer CV, 6 or fewer CV, 5 or fewer CV, 4 or fewer CV or 3 or fewer CV. The salt gradient may be, e.g., less than about 0.5 mM salt/column volume, between about 0.5 mM salt/column volume and about 5 mM salt/column volume, about 0.5 mM salt/column volume, 0.6 mM salt/column volume, 0.7 mM salt/column volume, 0.8 mM salt/column volume, 0.9 mM salt/column volume, 1 mM salt/column volume, 1.2 mM salt/column volume, 1.4 mM salt/column volume, 1.6 mM salt/column volume, 1.8 mM salt/column volume, 2 mM salt/column volume, 3 mM salt/column volume, 4 mM salt/column volume, 5 mM salt/column volume or greater than 5 mM salt/column volume.

### DETAILED DESCRIPTION

### Definitions

The terms “polypeptide” or “protein” are used interchangeably herein to refer to a polymer of amino acid residues. The terms also apply to amino acid polymers in which one or more amino acid residues is an analog or mimetic of a corresponding naturally occurring amino acid, as well as to naturally occurring amino acid polymers. The terms can also encompass amino acid polymers that have been modified, e.g., by the addition of carbohydrate residues to form glycoproteins, or phosphorylated. Polypeptides and proteins can be produced by a naturally-occurring and non-recombinant cell; or it is produced by a genetically-engineered or recombinant cell, and comprise molecules having the amino acid sequence of the native protein, or molecules having deletions from, additions to, and/or substitutions of one or more amino acids of the native sequence. The terms “polypeptide” and “protein” specifically encompass peptibody, domain-based proteins and antigen binding proteins, e.g., antibodies and fragments thereof, as well as sequences that have deletions from, additions to, and/or substitutions of one or more amino acids of any of the foregoing.

The term “antibody” refers to an intact immunoglobulin of any isotype, or an antigen binding fragment thereof that can compete with the intact antibody for specific binding to the target antigen, and includes, for instance, chimeric, humanized, fully human, and bispecific antibodies. An “antibody” as such is a species of an antigen binding protein. An intact antibody generally will comprise at least two full-length heavy chains and two full-length light chains. Antibodies may be derived solely from a single source, or may be “chimeric,” that is, different portions of the antibody may be derived from two different antibodies. The antigen binding proteins, antibodies, or binding fragments may be produced in hybridomas, by recombinant DNA techniques, or by enzymatic or chemical cleavage of intact antibodies.

The term “cation exchange material” or “cation exchange matrix” refers to a solid phase that is negatively charged and has free cations for exchange with cations in an aqueous solution passed over or through the solid phase. The charge may be provided by attaching one or more charged ligands to the solid phase, e.g. by covalent linking. Alternatively, or in addition, the charge may be an inherent property of the solid phase. Cation exchange material, matrix or resin may be placed or packed into a column useful for the purification of proteins.

The term “buffer” or “buffered solution” refers to solutions which resist changes in pH by the action of its conjugate acid-base range.

The term “loading buffer” or “equilibrium buffer” refers to the buffer containing the salt or salts which is mixed with the protein preparation for loading the protein preparation onto a chromatography matrix or column. This buffer is also used to equilibrate the matrix or column before loading, and to wash to matrix or column after loading the protein.

The term “wash buffer” is used herein to refer to the buffer that is passed over a chromatography matrix or column following loading of a composition or solution and prior to elution of the protein or isoform of interest. The wash buffer may serve to remove one or more contaminants or undesired isoforms from the chromatography matrix or column, without substantial elution of the desired protein or isoform.

The term “elution buffer” refers to the buffer used to elute the desired protein or isoform from a chromatography matrix or column. The pH and/or salt concentration of an elution buffer are typically different from the pH and/or salt concentration of the loading and/or wash buffer used to load or wash a particular column, to enable elution of the desired proteins from the column.

As used herein, the term “solution” refers to either a buffered or a non-buffered solution, including water.

The term “washing” a chromatography matrix means passing an appropriate buffer through or over the chromatography matrix.

The term “eluting” a molecule (e.g. a particular protein, isoform or contaminant) from a chromatography matrix means removing the molecule from such material, typically by passing an elution buffer over the chromatography matrix. The material is typically collected in aliquots or fractions as it is eluted from the matrix.

The term “neutral pH”, unless otherwise defined herein, refers to a pH of between 6.0 and 8.0, preferably between about 6.5 and about 7.5.

The term “mildly acidic”, when used in connection with a buffer, solution or the like, and unless otherwise defined herein, refers to a buffer or solution having a pH of between about 4.5 and about 6.5.

The term “acidic” or “low pH”, when used in connection with pH, a buffer, solution or the like, and unless otherwise defined herein, refers to a pH or a buffer or solution having a pH of between about 1 and about 6.5.

The term “contaminant” or “impurity” refers to any foreign or objectionable molecule, particularly a biological macromolecule such as a DNA, an RNA, or a protein, other than the protein being purified that is present in a sample of a protein being purified. Contaminants include, for example, other proteins from cells that secrete the protein being purified and proteins.

The term “separate” or “isolate” as used in connection with protein purification refers to the separation of a desired protein or isoform from a second protein or isoform or contaminant in a mixture comprising both the desired protein or isoform and a second protein or isoform or contaminant, such that at least the majority of the molecules of the desired protein or isoform are removed from that portion of the mixture that comprises at least the majority of the molecules of the second protein or isoform or contaminant. More specifically, the term “separate” or “isolate” is also used herein in connection with protein purification to refer to the separation of different structural isoforms of an IgG2 antibody, where the different structural isoforms are characterized by different disulfide bonding patterns.

The term “purify” or “purifying” a desired protein or isoform from a composition or solution comprising the desired protein or isoform and one or more contaminants or undesired isoform(s) means increasing the degree of purity of the desired protein or isoform in the composition or solution by removing (completely or partially) at least one contaminant (e.g., undesired isoform) from the composition or solution.

The term “to bind” or “binding” a molecule to an ion exchange material means exposing the molecule to the ion exchange material or matrix under appropriate conditions (e.g., pH and selected salt/buffer composition) such that the molecule is reversibly immobilized in or on the ion exchange material or matrix by virtue of ionic interactions between the molecule and a charged group or charged groups of the ion exchange material or matrix.

Cation Exchange Chromatography

Ion exchange chromatography separates compounds based on their net charge. Ionic molecules are classified as either anions (having a negative charge) or cations (having a positive charge). Some molecules (e.g., proteins) may have both anionic and cationic groups. A positively charged support (anion exchanger) will bind a compound with an overall negative charge. Conversely, a negatively charged support (cation exchanger) will bind a compound with an overall positive charge. Cation exchange media are known to those of skill in the art. Exemplary cation exchange media are described, e.g., in Protein Purification Methods, A Practical Approach, Ed. Harris ELV, Angal S, IRL Press Oxford, England (1989); Protein Purification, Ed. Janson J C, Ryden L, VCH-Verlag, Weinheim, Germany (1989); Process Scale Bioseparations for the Biopharmaceutical Industry, Ed. Shukla A A, Etzel M R, Gadam S, CRC Press Taylor & Francis Group (2007), pages 188-196; Protein Purification Handbook, GE Healthcare 2007 (18-1132-29) and Protein Purification, Principles, High Resolution Methods and Applications (2.sup.nd Edition 1998), Ed. Janson J-C and Ryden L, the disclosures of which are incorporated herein by reference in their entirety.

Ion exchange matrices can be further categorized as either strong or weak exchangers. Weak ion exchange matrices contain or are derived from a weak acid (such as a carboxymethyl group from, e.g., carboxylic acid (R—COO⁻), which gradually loses its charge as the pH decreases below 4 or 5. The ionic groups of exchange columns are covalently bound to the gel matrix and are compensated by small concentrations of counter ions, which are present in the buffer. A non-limiting example of the functional group in a WCX column would be —CH₂CH₂CH₂CO₂⁻.

Strong ion exchange matrices are charged (ionized) across a wide range of pH levels because they contain a strong acid (such as a sulfopropyl group) that remains charged from pH 1-14. Strong cation exchange (SCX) chromatography thus uses a resin with a functional group derived from a strong acid, such as sulfonic acid (R—SO3H). A non-limiting example of the functional group in a SCX column would be —CH₂CH₂CH₂SO₃⁻.

Cation exchange chromatography (CEX) has become a preferred method for IgG purification. The relatively mild buffer solution conditions of CEX help preserve the IgG native structure, while purifying it from host cell proteins and IgG variants (Shukla, et al., 2006). Optimal conditions for ion exchange chromatography of proteins are achieved when the pH of the elution buffer is within one pH unit of the pI of the protein. With average pI values of IgG molecules in the range of 7 to 8.5, the relatively mild buffers (from mildly acidic to neutral pH) provide optimal conditions for CEX of the positively charged (cation) IgG molecules. CEX is utilized as one of the downstream purification steps for recombinant IgG molecules by removing host cell protein (Chinese hamster ovaries cells) contaminants (Shukla, et al., 2006), deamidated species of the IgG molecule (Harris, et al., 2001; Basey, et al., U.S. Pat. No. 7,074,404), and other protein variants. Although CEX has become common practice in the biotech industry for the purification of IgG mAbs, purification of IgG2 disulfide isoforms by CEX has had only marginal success, especially at the analytical level.

IgG2 disulfide isoforms were previously enriched for biochemical and biophysical characterization using low pH (˜5) weak cation exchange (Wypych, et al., 2008). In general, CEX chromatography resolves relatively subtle chemical and structural differences in proteins based on differences in the overall surface charge of the molecule. For IgG2 disulfide isoforms, the three dimensional structure of each isoform creates a unique surface charge, allowing partial resolution by WCX chromatography. One of the challenges using this technique for disulfide isoform enrichment is that other post-translational modifications or protein variants (deamidation, aggregate, glycation, —C and —N terminal modifications, etc.) are often separated and enriched in the same collected fractions as the disulfide isoforms. Earlier studies identified WCX as the only non-denaturing chromatography technique capable separating the IgG2 disulfide isoforms on a semi-preparative-scale. Although WCX has been shown to work marginally well for producing moderately pure fractions of the “A” and “B” isoforms, this technique generally does a poor job in resolving the “A/B” and other sub-species of the disulfide isoforms. For example, IgG2 lambda antibodies have been shown to be composed primarily (>95%) of “A/B” and “A” isoforms (Wypych, et al., 2008; Dillon, et al., 2008), thereby making it especially difficult to resolve the low abundance “B” species using WCX.

Experiments detailed herein document the development of a new process, based on SCX, that resulted in increased yield of high purity IgG2 disulfide isoforms. The SCX process was then combined with additional novel separation technologies to augment the downstream purification process. Examples of commercial strong cation exchange (SCX) media useful with the methods of the present invention include GE Healthcare: SP-Sepharose FF, SP-Sepharose BB, SP-Sepharose XL, SP-Sepharose HP, Mini S, Mono S, Source 15S, Source 30S, Capto S, MacroCap SP, Streamline SP-XL, Streamline CST-1 (a multi-modal resin, but with a strong CEX component); Tosohaas Resins: Toyopearl Mega Cap TI SP-550 EC, Toyopearl Giga Cap S-650M, Toyopearl 650S, Toyopearl SP650S, Toyopearl SP550C; JT Baker Resins: Carboxy-Sulphon-5, 15 and 40 um, Sulfonic-5, 15, and 40 um; Applied Biosystems: Poros HS 20 and 50 um, Poros S 10 and 20 um; Pall Corp: S Ceramic Hyper D; Merck KGgA Resins: Fractogel EMD SO₃, Fractogel EMD SE Hicap, Fracto Prep SO₃; Biorad Resin Unosphere S. Additional sources of strong cation exchange chromatography materials include, e.g., Mustang S (available from Pall Corporation, East Hills, N.Y., USA), Partisphere SCX (available from Whatman plc, Brentford, UK), YMC-SCX 30 μm resin from YMC Co., Ltd., Allentown, Pa., and any cross-linked methacrylate modified with SO₃— groups, such as the Fractogel EMD SO₃ mentioned above.

Protein L

Protein L is a naturally occurring bacterial cell wall protein that shows specificity for IgG (Kastern, W., et al., (1990), Infect. Immun. 58, 1217-1222), similar to Protein A (Forsgren, A. and Sjoquist, J. (1966)) and Protein G (Bjorck, L. and Kronvall, G. (1984)). Although these proteins have been shown to bind with high affinity to IgG molecules, Protein L is unique in that it binds specifically to the light chain (LC) of IgG in close proximity to the Fab-Fc (hinge) interface. This is unlike Protein A and G which bind to the lower Fc portion of the heavy chains in the CH2-CH3 interface. Studies have shown that the major binding sites of Protein L are comprised within the variable domains of the IgG LC (Nilson, et al., 1992). More specifically, Protein L has been shown to only bind kappa LC of the VκI, VκIII, and VκIV subgroups. Experiments detailed herein indicate that Protein L is capable of differential binding to individual IgG2 disulfide isoforms.

In practicing the methods detailed herein, any of a number of different Protein L columns may be used, including, for example, the Pierce Protein L affinity resin from Thermo Scientific Pierce, Rockford, Ill.).

IgG2 Affinity Matrix

Antibodies are commonly developed against newly discovered proteins for use as immunoreagents. Multiple IgG2 specific clones were created and tested for domain specificity. Experiments performed in support of the present invention indicate that antibody HP-6014 (Harada, et al., 1991; Harada, et al., 1992) and antibodies having a similar epitope may be used to differentiate the IgG2 disulfide isoforms in connection with IgG2 antibody purification.

Integration

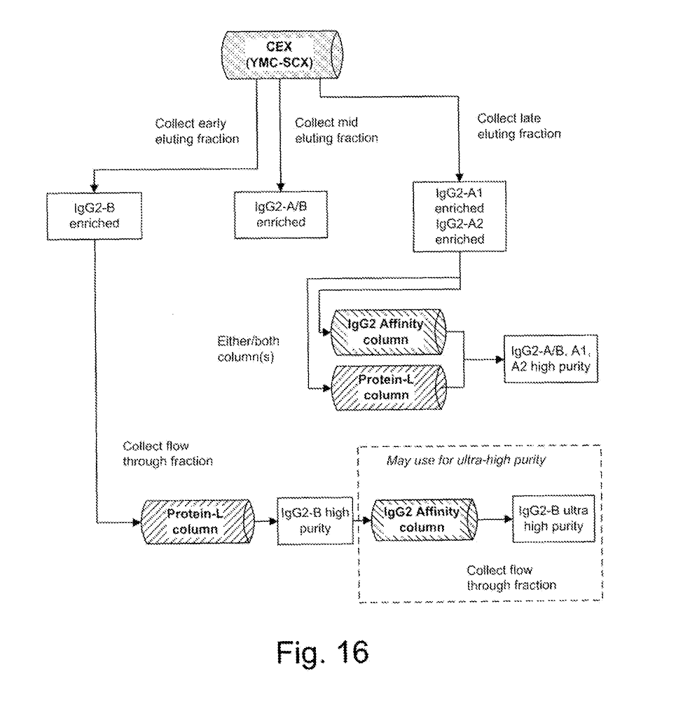

The methods described herein were developed for efficient separation of IgG2 disulfide isoforms utilizing SCX chromatography, a Protein L column, and/or a novel anti-human IgG2 isoform affinity column capable of separating IgG2 disulfide isoforms. These techniques have provided significant improvements in the purity of individual isoforms as well as a considerable increase in yield. For many characterization studies, it is desirable to have near 100% purity of the required variant. In the studies described herein, when using a single purification technique it was possible to obtain ˜75-85% purity for a desired isoform. When combining two or three of the techniques, it was possible to obtain 90-100% purity for the isoforms. The methods as detailed herein may be employed by one skill in the art to similarly purify isoforms of any IgG2 mAb. By way of example, the flow chart in FIG. 16 describes the combined application of these technologies to produce high purity fractions of mAb1 disulfide isoforms. Several different combinations of the three columns were implemented to improve purity of the desired IgG2 isoform (FIG. 16). Combinations of these individual separation techniques and columns have been successfully applied to a number of mAbs, including mAb 1, mAb2, mAb3, mAb4, mAb5, mAb7, mAb9, mAb10 and mAb11.

Further, such combinations may be applied by one of skill in the art to any IgG2 mAb. For example, a somewhat different approach was used in the case of mAb7, since Protein L was able to bind the VkII light chain containing mAb. As shown in Table 1, below, mAb7 used cation exchange as a starting column for purification of the B and A/β isoforms and the anti-hu IgG2 affinity column for the A isoform. Utilizing the different binding properties of each column allowed for extremely high purity material (95-100%) to be prepared at a relatively large scale. In summary, the multi-column strategy described herein has worked well for all IgG2 mAbs tested, but some method optimization may be performed by one of skill in the art when applying the methods to other IgG2 mAbs.

| | |
|---|---|
| — | |
| Purification | Purity by Reversed-phase (%) |

| Technique | B | A/B | A1 | A2 |
|---|---|---|---|---|
| mAb1 standard | 36 | 37 | 15 | 12 |
| process control | | | | |

| | | | |
|---|---|---|---|
| ProL | 10 | 7 | 83 |

| | | | | |
|---|---|---|---|---|
| CEX | 16 | 10 | 45 | 29 |
| CEX | 80 | 14 | 5 | 1 |

| | | | |
|---|---|---|---|
| ProL & CEX | 80 | 20 | 0 |

| | | | | |
|---|---|---|---|---|
| ProL & CEX | 1 | 0 | 57 | 42 |
| ProL & CEX | 83 | 11 | 5 | 1 |
| ProL & CEX | 14 | 71 | 7 | 8 |
| ProL & CEX | 0 | 0 | 68 | 32 |
| ProL & CEX | 0 | 0 | 52 | 48 |

| | | | |
|---|---|---|---|
| ProL & CEX | 9 | 82 | 9 |

| | | | | |
|---|---|---|---|---|
| ProL/CEX/Anti-IgG2 | 0 | 0 | 84 | 16 |
| ProL/CEX/Anti-IgG2 | 0 | 5 | 9 | 86 |
| ProL/CEX/Anti-IgG2 | 0 | 0 | 84 | 16 |
| ProL/CEX/Anti-IgG2 | 0 | 0 | 86 | 14 |
| ProL/CEX/Anti-IgG2 | 0 | 5 | 7 | 88 |

Table 2, below, shows a qualitative summary of data described herein in a format that can be referenced for general IgG2 disulfide isoform binding properties.

TABLE 2
Human IgG1, IgG2 and IgG2 disulfide isoforms recognition
specificity for cation exchange chromatography
(CEX) and different proteins and antibodies.

| IgG2 recognition and separation | IgG2- | IgG2- | IgG2- | IgG2- |
|---|---|---|---|---|
| specificity for different agents | A1 | A2 | A/B | B |
| Cation Exchange | | | | |
| Weak CEX | ++ | ++ | + | + |
| Strong high-capacity CEX | +++ | ++++ | ++ | + |
| Protein L | | | | |
| Protein L from Peptostreptococcus magnus | +++ | ++++ | ++ | +/− |
| Protein A from Staphylococcus aureus, SpA | +++ | +++ | +++ | +++ |
| Protein G from Staphylococcus aureus, SpG | +++ | +++ | +++ | +++ |
| Affinity: | | | | |
| Murine anti-human IgG2 mAb HP-6014 | ++++ | +/− | +/− | − |
| Murine anti-human IgG2 mAb HP-6002 | +++ | +++ | +++ | +++ |

+/− binding and non-bind have been observed for different IgG'2s

One of the applications or uses of the described techniques is to obtain highest purity isoforms for assessment of their potency and other parameters. Another application or use is to obtain bulk material with predetermined, defined percentages of the isoforms. This is useful to better enable comparability of the bulk materials for clinical trials, commercial use and different production processes. For example, the methods described herein may be used in connection with large preparative scale cation exchange columns (Shukla et al., 2006; Shukla et al., 2004) in downstream processing during mAb production, with a goal of controlling the relative abundances of the IgG2 disulfide isoforms. In order to produce bulk material with defined percentages of isoforms, the purification process may result in collecting limited CEX fractions. The cut-off time or cut-off elution volume may be adjusted after, e.g., an on-line measurement of the isoform abundances by RP-HPLC assay. The combined use of, e.g., a rapid RP-HPLC assay (e.g., 2-3 minute runs) and CEX during the downstream purification process is one way to implement a manufacturing control for IgG2 isoforms.

Methods of the present invention may be utilized during production to separate and purify individual IgG2-A and IgG2-B disulfide isoforms, e.g., on gram and kilogram scales. The methods may be also utilized to recognize and measure abundances of the individual IgG2 isoforms, e.g., in blood from patients, for diagnostic purposes on nanogram and microgram scale. In addition, different disulfide isoforms (e.g., B, A/B, A1, A2) may be isolated so that potency, stability, propensity to aggregate, other characteristics of the disulfide isoforms, can be assessed, e.g., during protein production.

The overall disulfide isoforms ratio of a mAb (e.g., drug substance) may be modified, e.g., as follows: (a) starting collection later in the cation exchange elution peak to shift ratio to less B form and more A1, A2 forms +A/B form; (b) stopping collection earlier in the cation exchange elution peak to shift ratio to less A1, A2 forms and more B form +A/B form; (c) starting collection later and stopping collection earlier, collecting the middle portion of the cation exchange elution peak, to have more A/B form and less of the B, A1 and A2 forms; and/or collecting and pooling the front and back fractions of the cation exchange elution peak, to have less A/B form and more B, A1 and A2 forms.

Redox reagents added to mAb in solution may also be removed to change the ratio of disulfide isoforms, by binding the mAb to the cation exchange resin, washing to remove remaining unbound redox reagents, then eluting. Alternatively or in addition, mAb can be bound to the cation exchange resin, washed with redox reagents under buffer conditions to allow changes in disulfide isoforms, then washed to remove the redox reagents and finally eluted.

### Adapting Preparative Scale SCX Methods to Different Antibodies

Preparative scale production may include columns having greater than, e.g., 5 ml volume, larger resin bead size (e.g., 30 micron beads), larger diameter columns (e.g., 7 cm or greater), higher flow rates (e.g., ˜100 cm/hr), greater loading (e.g., >2 g/L, >12 g/L), a single, relatively short (e.g., 10 CV or less), and/or shallow to very shallow (e.g., 1 to 2 mM salt/column volume) gradient of increasing salt for the peak elution. In some embodiments, preparative scale production is characterized by single, relatively short, shallow gradient of increasing salt and higher resin loading (e.g., >2 g mAb/L, >4 g mAb/L, >6 g mAb/L, >8 g mAb/L, >10 g mAb/L, >12 g mAb/L, >15 g mAb/L, or >30 g mAb/L).

As suggested in Examples 10 and 11, the elution gradient employed in a preparative scale application of the invention may be optimized for different mAbs. The following describes a method by which this can be accomplished. The gradient may initially be scouted on a bench-scale (e.g., 1 cm diameter or smaller, with similar bed height to the preparative column) cation exchange column, using a pH 5.0 to 5.2 buffer with no salt added as buffer A, and a pH 5.2 to pH 4.5 buffer with 250 mM or 400 mM NaCl or greater added as buffer B. The pH of the buffer A is intended to be similar to the pH of the monoclonal antibody load (drug substance or earlier in-process pool) and could be somewhat higher or lower than pH 5 if necessary or desired. After equilibration with A buffer, the scout column may be loaded with 0.5 to 2 mg mAb per mL of packed resin bed. The mAb may be diluted as needed with A buffer to a volume convenient for loading, e.g., one column volume (CV), or as needed to reduce conductivity to allow binding to the cation exchange resin. After loading, the scout column may be washed briefly (1 to 3 CV) with buffer A, then a long gradient (20 to 50 CV) from 0% to 100% B (or 10% to 90% B or 20% to 80% B) may be applied to the column. If the long gradient is to be started at a % B greater than 0%, the wash after loading in the preceding step is typically run as a short gradient (1 to 3 CV) from 0% B to the desired starting % B for the long gradient, for example, from 0% B to 10% B over 2 CV for a long gradient that will start at 10% B.

Detection of the mAb peak elution is by 280 nm absorbance. The % B buffer at which the mAb begins to elute is set as the beginning of the elution gradient for the preparative column. The % B buffer at which most or all of the mAb has eluted (the tailing side of the peak) is set as the ending % B for the preparative column elution gradient. The aim is to determine a shallow gradient of about 1 to 2 mM NaCl per CV for the preparative column elution. If desired after the long elution scouting run, a test run of the preparative elution gradient may be run on the scout column, using the same buffers and loading as for the first scouting run described above. In the test run, after loading, a short wash (1 to 3 CV) from 0% B to the target starting % B is run, followed by a 10 CV gradient (which could be shorter or longer as desired) from the starting % B to the ending % B for the elution of the mAb, to give a gradient of about 1 to 2 mM NaCl per CV. If isocratic elution is desired, that elution buffer strength could also be determined from the scouting gradient. It is possible that for some mAbs and conditions a steeper gradient (greater than 2 mM NaCl per CV) would also provide the desired resolution of disulfide isoforms.

The preparative column can be loaded from about 2 g of mAb per L of packed resin to 12 g of mAb per L of packed resin or more, for example, depending on the characteristics of the mAb or the resolution of disulfide isoforms required, the column could be loaded with a greater amount of mAb (such as 30 g/L or more), or the column could be cycled. The preparative column is equilibrated and run with the same A and B buffer compositions as was used in the long gradient scouting and test gradient runs. In general, the column is first pre-equilibrated with some volumes of 100% B buffer, then equilibrated with sufficient 100% A buffer. The mAb load is diluted with A buffer to a volume convenient for liquid handling or to a low enough conductivity for binding to the cation exchange resin. The mAb load may also be prepared for loading by other means such as Ultrafiltration/diafiltration for buffer exchange if needed or preferred. After loading, the column is washed with a 1 to 3 CV gradient from 0% B buffer to the target starting % of B buffer for the elution. Then the elution gradient determined in the long gradient scouting or test run may be applied to the column. This gradient is generally about 1 to 2 mM of NaCl per column volume. Elution of the mAb is detected by absorbance at 280 nm. Fractions may be collected for later assay by RPHPLC for the disulfide isoforms, or start and stop of collection may be controlled by A280 nm or by PAT. If desired, specific fractions may be diluted with A buffer and re-applied to the cation exchange column for further enrichment of a particular disulfide isoform.

### CITED REFERENCES

Basey, C. D. and Blank, G. S. Protein purification. [U.S. Pat. No. 7,074,404]. 7-11-2006. 9-24-2004.
Ref Type: Patent
Bjorck, L. and Kronvall, G. (1984). Purification and some properties of streptococcal protein G, a novel IgG-binding reagent. J. Immunol. 133, 969-974.
Bondarenko, P. V., Dillon, T. M., Wiltzius, J., and Chou, R. Methods of recognition and separation of IgG2 disulfide isoforms. [Amgen Invention Disclosure D-2421]. 2010.
Ref Type: Patent
Chemey, B. (2010). Comparability of biotechnology derived protein products: lessons from the U.S. experience. WCBP A presentation from a Deputy Director of FDA.
Dillon, T. M., Bondarenko, P. V., Pipes, G. D., Ricci, M., Rehder, D. S., and Kleemann, G. K. LC/MS method of analyzing high molecular weight proteins. [U.S. Pat. No. 7,329,353, WO2005073732, Provisional Application filed Jan. 21, 2004]. 2-12-2008a.
Ref Type: Patent
Dillon, T. M., Bondarenko, P. V., Rehder, D. S., Pipes, G. D., Kleemann, G. R., and Ricci, M. S. (2006a). Optimization of a reversed-phase LC/MS method for characterizing recombinant antibody heterogeneity and stability. J. Chromatogr. A 1120, 112-120.
Dillon, T. M., Rehder, D. S., Bondarenko, P. V., Ricci, M., Gadgil, H. S., Banks, D., Zhou, J., and Lu, Y. Methods for refolding of recombinant antibodies. [Patent application WO2006047340, US20060194280, CA2584211, AU5299716, EP1805320]. 5-24-2006b.
Ref Type: Patent
Dillon, T. M., Speed-Ricci, M., Vezina, C., Flynn, G. C., Liu, Y. D., Rehder, D. S., Plant, M., Henkle, B., Li, Y., Varnum, B., Wypych, J., Balland, A., and Bondarenko, P. V. (2008b). Structural and functional characterization of disulfide isoforms of the human IgG2 subclass. J. Biol. Chem. 283, 16206-16215.
Forsgren, A. and Sjoquist, J. (1966). “Protein A” from S. aureus. I. Pseudo-immune reaction with human gamma-globulin. J. Immunol. 97, 822-827.
Harada, S., Hata, S., Kosada, Y., and Kondo, E. (1991). Identification of epitopes recognized by a panel of six anti-human IgG2 monoclonal antibodies. J. Immunol. Methods 141, 89-96.
Harada, S., Takagi, S., Kosada, Y., and Kondo, E. (1992). Hinge region of human IgG2 protein: conformational studies with monoclonal antibodies. Mol. Immunol. 29, 145-149.
Harris, R. J., Kabakoff, B., Macchi, F. D., Shen, F. J., Kwong, M. Y., Andya, J. D., Shire, S. J., Bjork, N., Totpal, K., and Chen, A. B. (2001). Identification of multiple sources of charge heterogeneity in a recombinant antibody. J. Chromatogr. B Biomed. Sci. Appl. 752, 233-245.
Kastern, W., Holst, E., Nielsen, E., Sjobring, U., and Bjorck, L. (1990). Protein L, a bacterial immunoglobulin-binding protein and possible virulence determinant. Infect. Immun. 58, 1217-1222.
Nilson, B. H., Solomon, A., Bjorck, L., and Akerstrom, B. (1992). Protein L from Peptostreptococcus magnus binds to the kappa light chain variable domain. J. Biol. Chem. 267, 2234-2239.
Reimer, C. B., Phillips, D. J., Aloisio, C. H., Moore, D. D., Galland, G. G., Wells, T. W., Black, C. M., and McDougal, J. S. (1984). Evaluation of thirty-one mouse monoclonal antibodies to human IgG epitopes. Hybridoma 3, 263-275.
Shukla, A. A., Hubbard, B., Tressel, T., Guhan, S., and Low, D. (2006). Downstream processing of monoclonal antibodies-Application of platform approaches. J. Chromatogr. B.
Silverman, J., Liu, Q., Bakker, A., To, W., Duguay, A., Alba, B. M., Smith, R., Rivas, A., Li, P., Le, H., Whitehorn, E., Moore, K. W., Swimmer, C., Perlroth, V., Vogt, M., Kolkman, J., and Stemmer, W. P. (2005). Multivalent avimer proteins evolved by exon shuffling of a family of human receptor domains. Nat. Biotechnol. 23, 1556-1561.
Wypych, J., Li, M., Guo, A., Zhang, Z., Martinez, T., Allen, M., Fodor, S., Kelner, D., Flynn, G. C., Liu, Y. D., Bondarenko, P. V., Speed-Ricci, M., Dillon, T. M., and Balland, A. (2008). Human IgG2 antibodies display disulfide mediated structural isoforms. J. Biol. Chem. 283, 16194-16205.

The following examples, including the experiments conducted and the results achieved, are provided for illustrative purposes only and are not to be construed as limiting the scope of the appended claims.

### Example 1

### Comparison of WCX and SCX Resins for Enrichment of mAb1 IgG2 Isoforms Using Analytical Columns

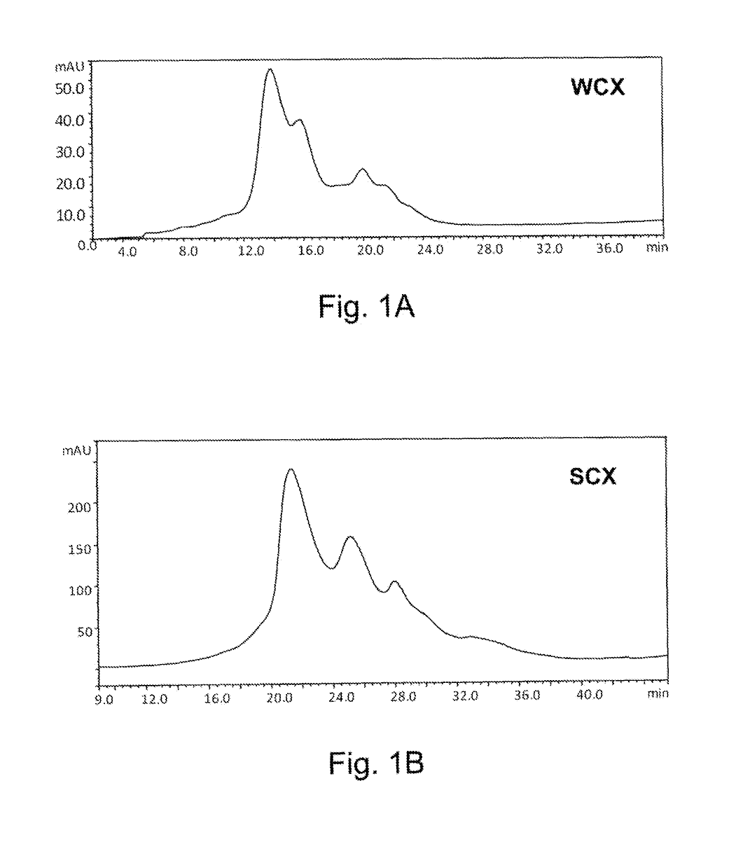

A comparison between a standard WCX column (ProPac WCX-10, 250×4.6 mm, P/N 54993, Dionex Corp., Sunnyvale, Calif.) and an SCX column (BioPro-SP, 100×4.6 mm, 5 um, P/N SF00S5-1046WP, YMC Co., Ltd., Allentown, Pa.) was made using low pH (˜5) buffers run on an Agilent 1100 HPLC analytical system. The protein was loaded at pH˜5 and eluted using a salt and pH gradient. The SCX resin was able to provide improved resolution of the mAb1 disulfide isoforms using these analytical scale columns (FIG. 1). In fact, the superior separation of IgG2 disulfide isoforms was achieved using a 4.6×100 mm YMC-SCX column with 5 μm resin versus a 4.6×250 mm Dionex column with 10 μm resin. This was a surprising and unexpected result since longer columns are typically expected to improve chromatographic resolution of biomolecules. In the present example, the shorter (100 mm) column with SCX resin showed superior results as compared with the longer (250 mm) column with WCX resin. This was attributed to higher capacity and greater separation power of the SCX column YMC BioPro SP-F as compared to the previously described WCX resin.

### Example 2

### CEX Recognition and Separation of IgG2 Disulfide Isoforms Using Preparative Column

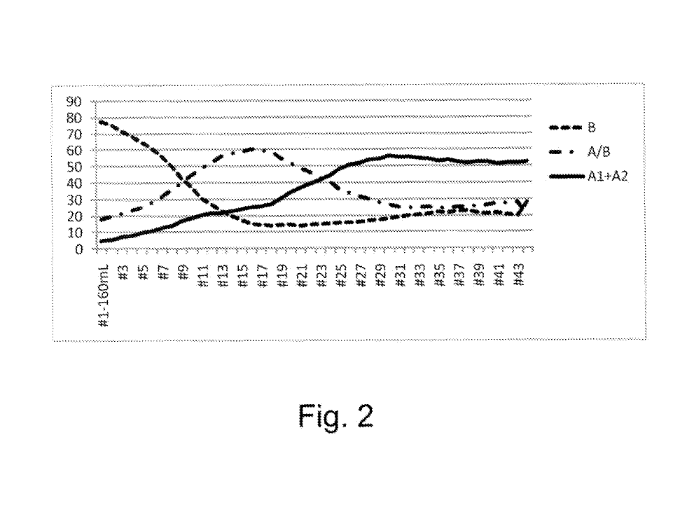

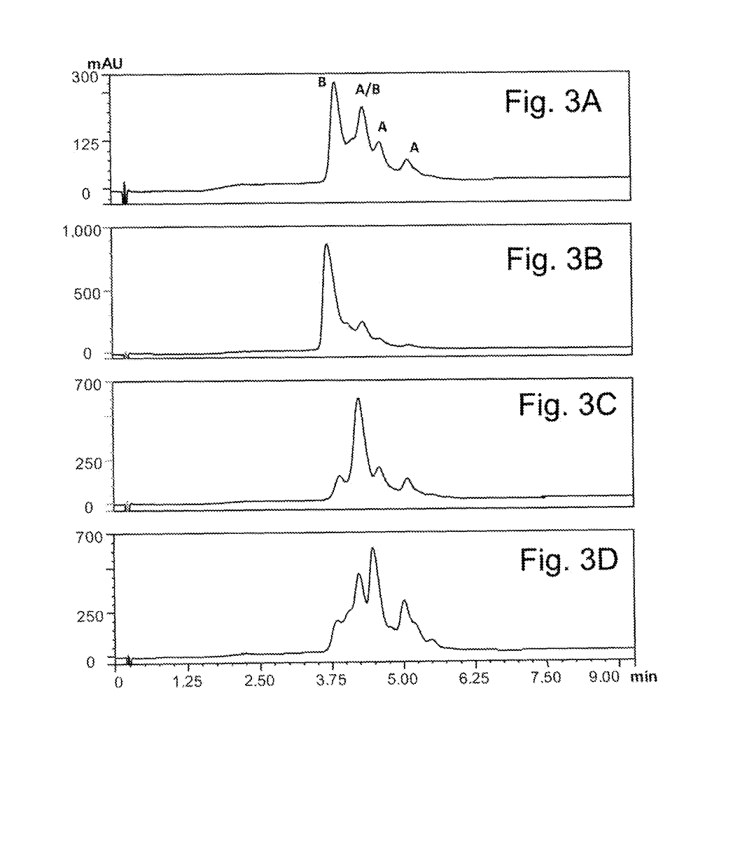

To assess if the SCX resin could provide similar resolution of IgG2 disulfide isoforms at a preparative scale, a larger 500 mL FPLC column was packed using YMC-SCX 30 μm resin (YMC-BioPro S30, P/N SPA0S30, YMC Co., Ltd., Allentown, Pa.). The protein was loaded using an approximate salt concentration of 100 mM NaCl and pH 5.2. A gradient elution was then used which increased the salt concentration to ˜250 mM and lowered the pH to ˜4.5. The IgG2-β isoform of mAb1 was least retained and eluted first from the column (FIG. 2). As the salt concentration increased and the pH decreased, IgG2-AB began eluting followed by IgG2-A. The collected cation exchange fractions were analyzed by a reversed-phase HPLC assay to show significant enrichment of disulfide isoforms by the FPLC column with YMC-SCX resin (FIG. 3). Reversed-phase analysis has been shown to resolve the IgG2 disulfide isoforms based on their hydrophobic properties. Generally, four distinct peaks are observed for reversed-phase of IgG2. The elution order of the disulfide isoforms from reversed-phase is IgG2-B (Peak-1), IgG2-A/B (Peak-2), and IgG2-A (Peak-3 & 4). Although Peaks-3 and -4 have been shown to contain the same inter-chain disulfide connectivity, the existence of two species by reversed-phase is thought to be a result of minor differences in the core hinge structure. In this description, the two IgG-A species were differentiated by denoting them as A1 and A2, relative to their NR-RP elution order (FIG. 3). It is believed that this is the first example of an IgG2 disulfide isoform enriched to greater than 50% purity using preparative scale CEX and gram level IgG2 loading.

### Example 3

### Protein L Recognition and Separation of IgG2 Disulfide Isoforms—5 Ml Semi-Preparative Column

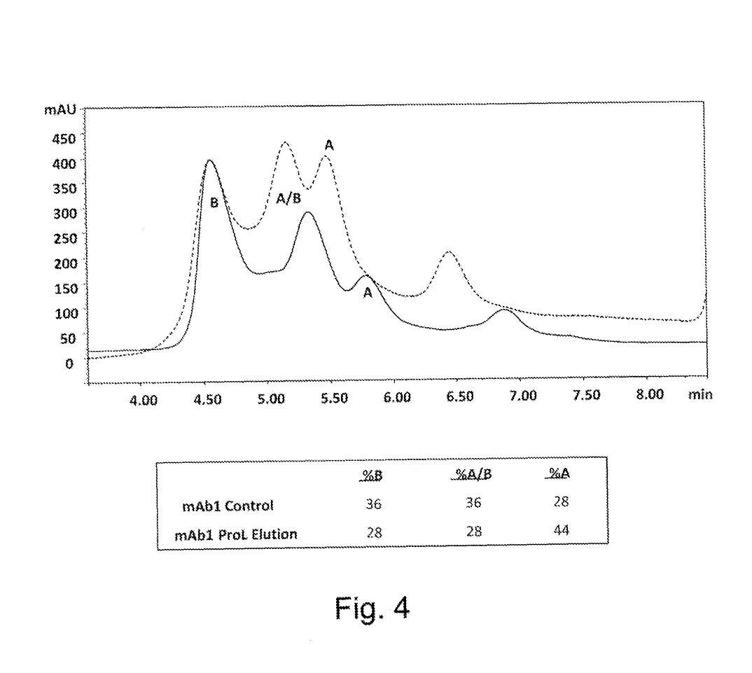

A 5 mL Pierce Chromatography Cartridge (PN-89929) was purchased and installed on an Agilent 1100 HPLC. The running buffers were PBS pH 7.2 (loading) and 100 mM glycine pH 2.8 (elution). Samples were diluted in PBS (1:1) and injected onto the column. The protein was loaded at pH 7.2 and eluted using a pH gradient. The 5 mL Protein L column was able to provide enrichment of the mAb1 disulfide isoforms as monitored by reversed-phase HPLC (FIG. 4). The IgG2-A species were more strongly retained by Protein L, requiring a lower pH buffer for elution.

### Example 4

### Protein L Recognition and Separation of IgG2 Disulfide Isoforms—24 Ml Preparative Column

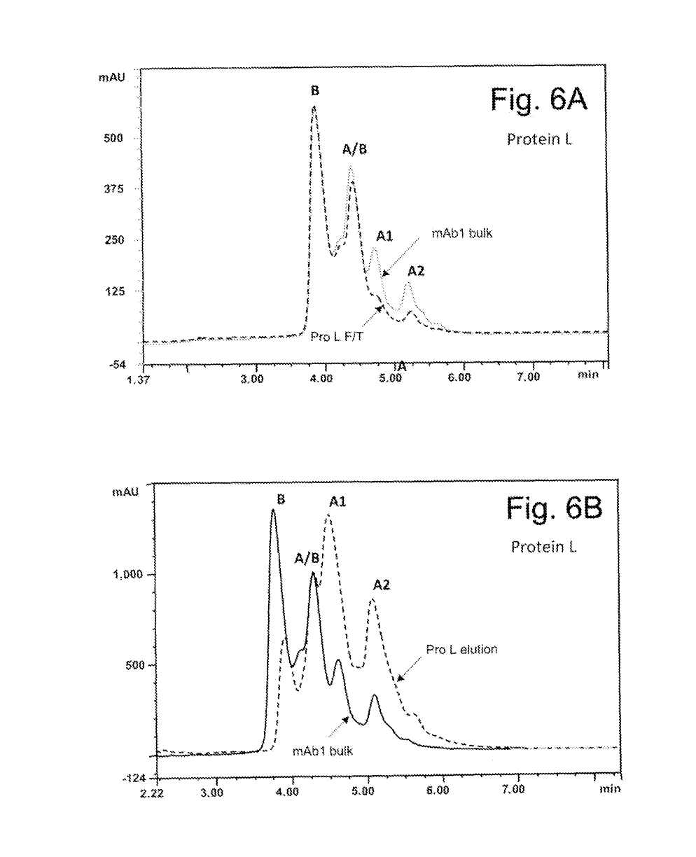

Pierce Protein L affinity resin (24 mL) was purchased (Protein L Agarose, P/N 20512, Thermo Scientific Pierce, Rockford, Ill.) and packed into a XK16/20 column (Bio LC column, P/N 19-0315-01, GE Healthcare, Pittsburgh, Pa.). The column was installed on an AKTA FPLC system and utilized by using the buffers listed above. In one application, mAb1 material was loaded on a 24 mL Protein L column, eluted with monitoring by UV detection at 214 & 280 nm. A large portion of the material was not retained and was washed through with the running buffer. A smaller portion of the mAb1 material was retained and later eluted using a pH gradient from pH 7.2→2.8. Fractions were collected across the Protein L separation and analyzed by reversed-phase (FIG. 6). As previously observed with the 5 mL Protein L column, the IgG2-A species were retained and later eluted as the pH was lowered using the 24 mL column (FIG. 6B). Likewise, the flow through (F/T) fractions showed depletion of the mAb1 IgG2-A species and therefore enrichment of IgG2-B material (FIG. 6A). The IgG2-A/B isoform was found in both F/T and eluted fractions, displaying intermediate binding properties.

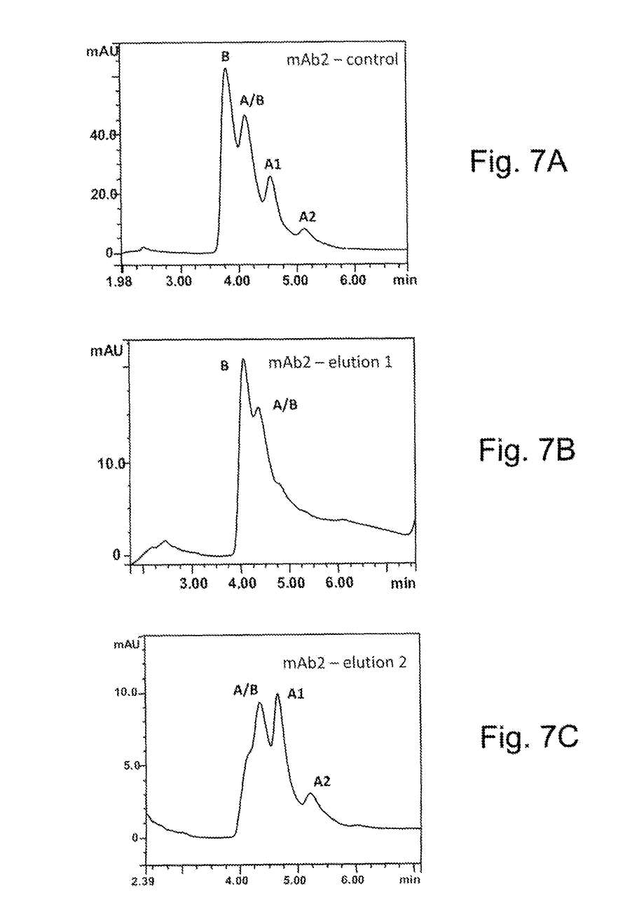

mAb2 was also tested using the same separation procedure, as described above for mAb1. mAb2 showed binding to Protein L, but, unlike mAb1, all isoforms were retained. mAb2 material began eluting from the Protein L column at approximately pH 4 (FIG. 7B), with a later fraction eluting at approximately pH 3 (FIG. 7B). The fractions were analyzed by reversed-phase HPLC, showing the same trend of elution as mAb1. IgG2-B was least retained followed by IgG2-A/B, with IgG2-A species showing the highest affinity for Protein L.

### Example 5

### Protein L Recognition and Separation of IgG2 Disulfide Isoforms—300 Ml Large Preparative Column

A larger scale Protein L column (˜300 mL) was packed and tested again using mAb1 & mAb2, showing comparable results. This larger column format provided a larger loading capacity of >100 mg load, while effectively separating the IgG2 disulfide isoforms. mAb3 (IgG2-VκI) was another IgG2 molecule used to test the larger scale Protein L column.

### Example 6

### Selection of Optimal Buffer for Protein L Purification

This example demonstrates approaches for selecting buffers for purifying and eluting desired disulfide isoforms in connection with the above-described Protein L method. For example, one goal was to have the IgG2-B form for mAb2 not bind, so it could be collected in the flow-through. Another goal was to have a higher pH elution for the IgG2-A species, as observed for mAb1.

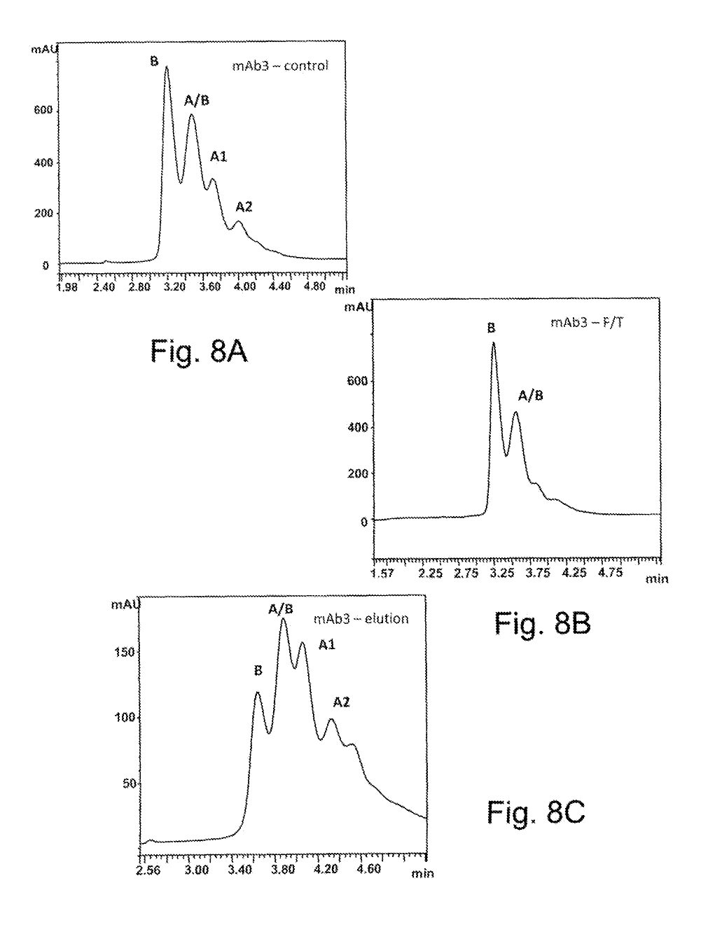

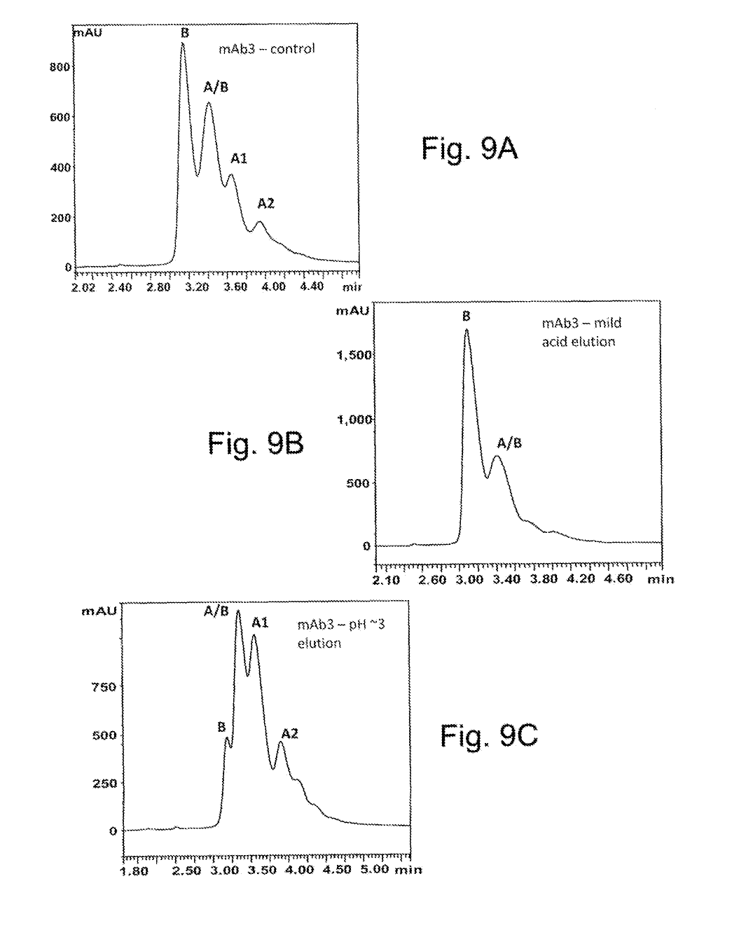

Different running buffers were evaluated in an attempt to adjust the binding and elution properties of the individual isoforms and reduce non-specific protein interactions with the column matrix resulting in peak tailing. For example, it was desired to not have the IgG2-β isoform of mAb2 retained on the Protein L column and therefore collected in the flow through. This allowed for partial removal of this isoform from the elution material. In addition, it was desired to use a higher pH elution for the IgG2-A species, as extended time at the low pH can cause irreversible denaturation of the protein. The optimal buffer selection for mAb3 was: A) 25 mM MOPS, pH 6.5; B) 100 mM glycine, pH 2.8. By using these buffers, good isoform resolution was observed for mAb3 (FIG. 8), but the lower pH elution was still not optimal. Therefore, Gentle Ag/Ab Binding and Elution buffers with proprietary compositions were purchased from Pierce and tested using mAb3. The buffers were listed to provide near neutral pH elution from Protein L columns. As shown in FIG. 9, the Gentle Ag/Ab Binding and Elution buffers were able to provide higher pH binding and elution from the Protein L column with good purity of isoform fractions. A disadvantage of the Gentle Ag/Ab Binding and Elution buffers was higher FPLC system back pressure.

### Example 7

### Affinity Recognition and Separation Using Antibodies Specific to IgG2 Disulfide Isoforms and SEC

Mouse anti-human IgG2 mAb clones HP-6002 (Fc specific; Abcam, Cambridge, Mass.) and HP-6014 (F(ab)2 specific; Acris Antibodies Inc., San Diego, Calif.), were tested for their ability to bind specific IgG2 disulfide isoforms of mAb7, mAb2, and mAb1.

Fractions of the enriched IgG2 disulfide isoforms obtained using the redox refolding method according to U.S. Pat. No. 7,928,205 (IgG2-A (˜65%) and IgG2-B (˜65%)), as well as non-enriched control IgG2 material were compared. The IgG2 samples were diluted in PBS and mixed with the anti-human IgG2 mAbs at an approximate 1:2.5 molar ratio. This ratio was chosen to provide at least two anti-human mAbs for a single IgG2 molecule.

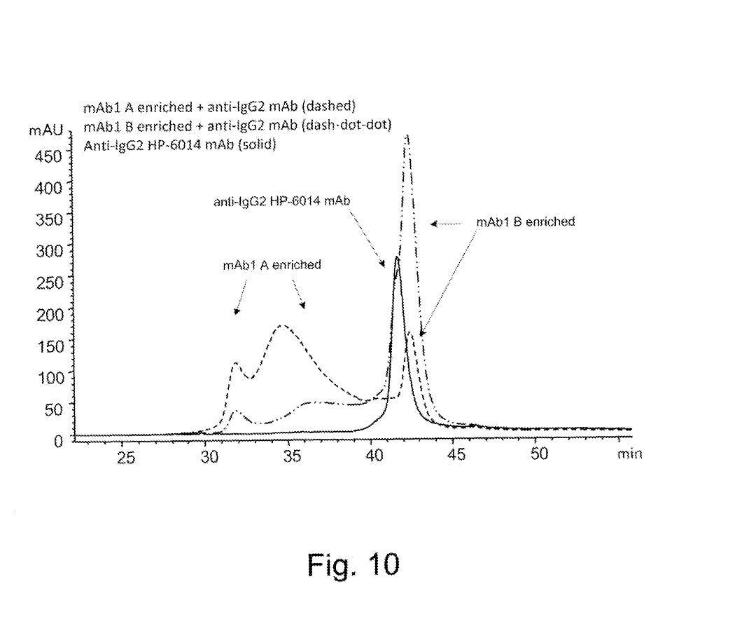

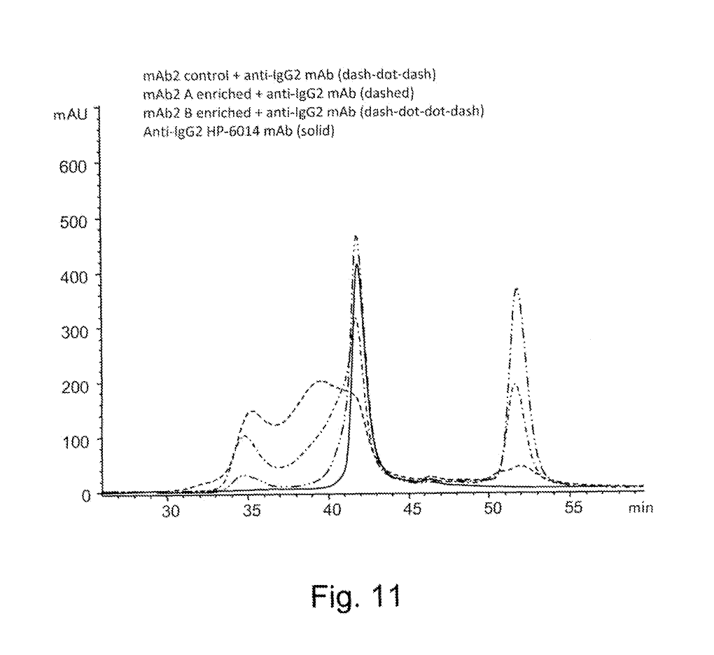

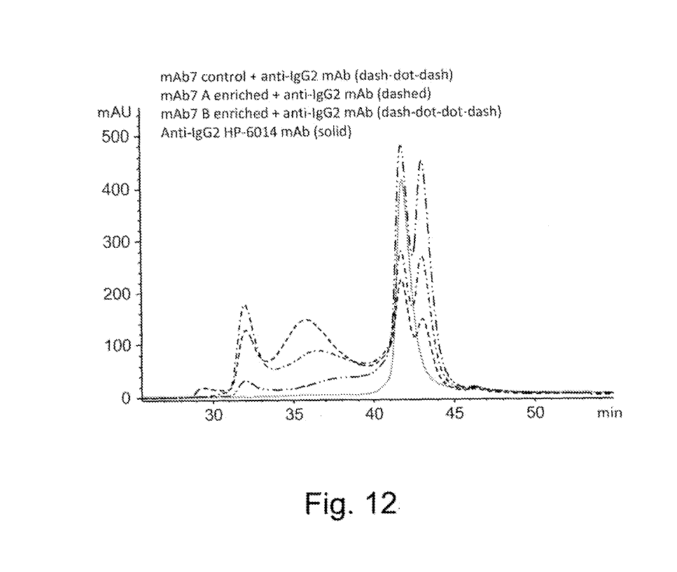

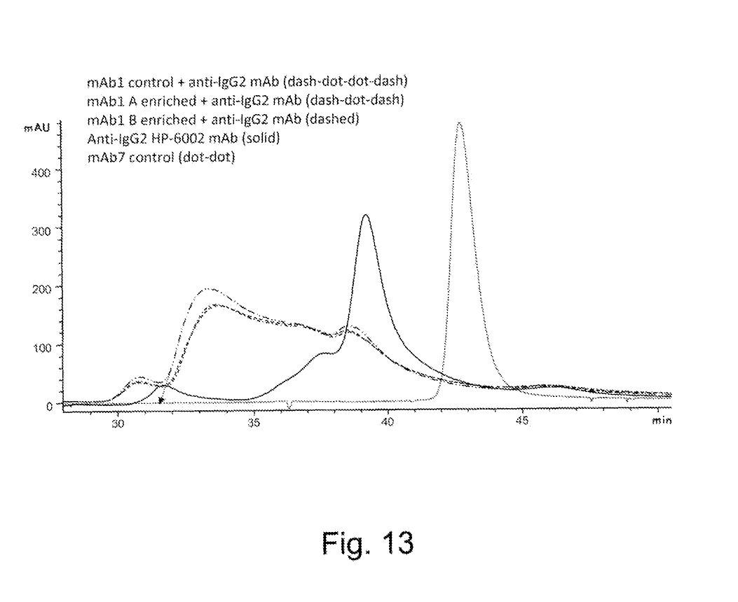

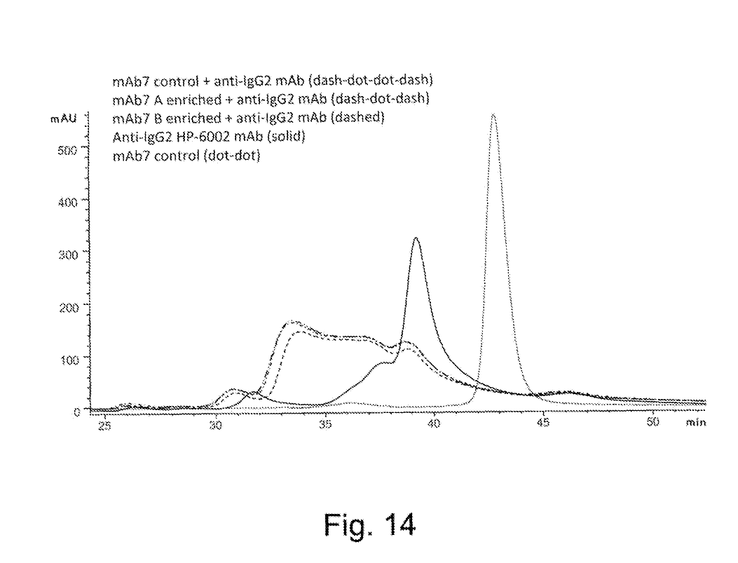

The samples were allowed to react for at least 1 hour prior to analysis. Samples were analyzed using size exclusion chromatography (SEC). In SEC, larger species elute earlier from the column due to decreased interactions with the pores of the stationary phase. Therefore, complexes of IgG2 molecules and mouse anti-human IgG2 eluted earlier than monomeric IgG2 molecules that did not interact with the mouse antibody. The SEC binding experiments using clone HP-6014 revealed the specificity of this clone for the IgG2-A species (FIGS. 10-12) and indicated non-binding of IgG2-B species. On the other hand, clone HP-6002 was unable to differentiate the disulfide isoforms as shown by the similar SEC profiles when IgG2-A and IgG2-B enriched materials were incubated with HP-6002 (FIGS. 13-14). SEC resolved the early eluting complexed species from late eluting IgG monomer as follows. When IgG2-A material of mAb2, mAb7, and mAb1 were incubated with HP-6014, the amount of complexed species (FIG. 10-14, eluting at 30-40 minutes) increased relative to HP-6014 incubation with control IgG2 material. When the IgG2-B materials were incubated with HP-6014, a significant decrease in complexed species was observed relative to HP-6014 incubation with control IgG2 material.

These results indicate little to no binding of IgG2-B to HP-6014 and strong binding of IgG2-A to HP-6014. The binding properties of IgG2-A/B were not ascertained from these data. An enriched IgG2-A/B fraction was not available to study because this IgG2 isoform had not been prior enriched by the redox procedure (Dillon et al., 2006b; Dillon et al., 2008b). Incubations of all samples with HP-6002 showed equivalent binding and therefore no specificity for the IgG2 disulfide isoforms (FIGS. 13-14).

### Example 8

### HP-6014 Affinity Column Purification

To further test the ability of HP-6014 anti-human IgG2 for separating IgG2 disulfide isoforms, an affinity column was prepared using the manufacturer's protocol as follows. 18 mL of Affinity-Gel 15 (Active Ester Agarose 25 mL, Bio-Rad Labs Cat#153-6051) activated with immobilized HP-6014 were placed in a 50 ml tube and exchanged with cold DI water three times to remove any potential residual preservatives. The column matrix was washed twice with 32 mL of 25 mM HEPES, pH 8.0, and resuspended (mixing well) with 15 mL of 25 mM HEPES, pH 8.0.

One set of columns was prepared with anti-Hu IgG2-UNLB (Clone HP6014; Southern Biotech Cat#9080-01), 0.5 mg/mL×20 ea=10 mg/20 mL in total. Another set was prepared with Hybridoma Reagent Laboratory HP6014P (mouse IgG1 mAb anti-human IgG2 Fab) as 2 mg/mL×18 vial=36 mg/18 mL in total.

The reactions were carried out in a total of 51 mL, including 18 mL of Affi-15 Gel +15 mL of 25 mM HEPES (pH8.0)+36 mg/18 ml anti-Hu IgG2, and were incubated at 4° C. for two hours (with occasional mixing). The mixture was allowed to stand at ambient temperature for another two hours (with occasional mixing). The coupling efficiency was monitored at each step by rapid (10 minute gradient time), high throughput RP-HPLC. The reaction was then quenched with 0.1 M Tris, pH8.0. Representative data are shown below.

| Time | Area | coupling, % |
|---|---|---|
| 0 hr | 114.0 | |
| 4 C. for 2 hr | 13.36 | 88.3 |
| 4 C. for 2 hr+ Rm temp for 1 hr | 4.93 | 95.7 |
| 4 C. for 2 hr+ Rm temp for 2 hr | 3.68 | 96.8 |
| Quenching− End | 3.27 | 97.1 |

The columns were then packed and rinsed with DPBS at pH 7.2 for ˜1 hour prior to use.

Murine Monoclonal Anti-Human IgG2 (clone HP-6014; Sigma-Aldrich cat. no. 15635) was coupled to Affi-Gel chromatography media as described above and packed in a XK16/20 column (Bio LC column, P/N 19-0315-01, GE Healthcare, Pittsburgh, Pa.). The column was installed on an AKTA FPLC system (GE Healthcare, Pittsburgh, Pa.) and run at room temperature. Samples were diluted in PBS pH 7.2 (1:1), injected onto the column and washed using PBS until a stable baseline was reached. The protein was eluted using a pH step gradient (100 mM glycine pH 2.8). The pH of the collected fractions was raised to >5.0 immediately following elution using an appropriate buffer. The purity of the fractions was tested using reversed-phase HPLC analysis (Dillon et al., 2008a; Dillon et al., 2006a).

### Example 9

### Affinity Recognition and Separation Using Immobilized Antibodies Specific to IgG2 Disulfide Isoforms—24 mL Preparative Column

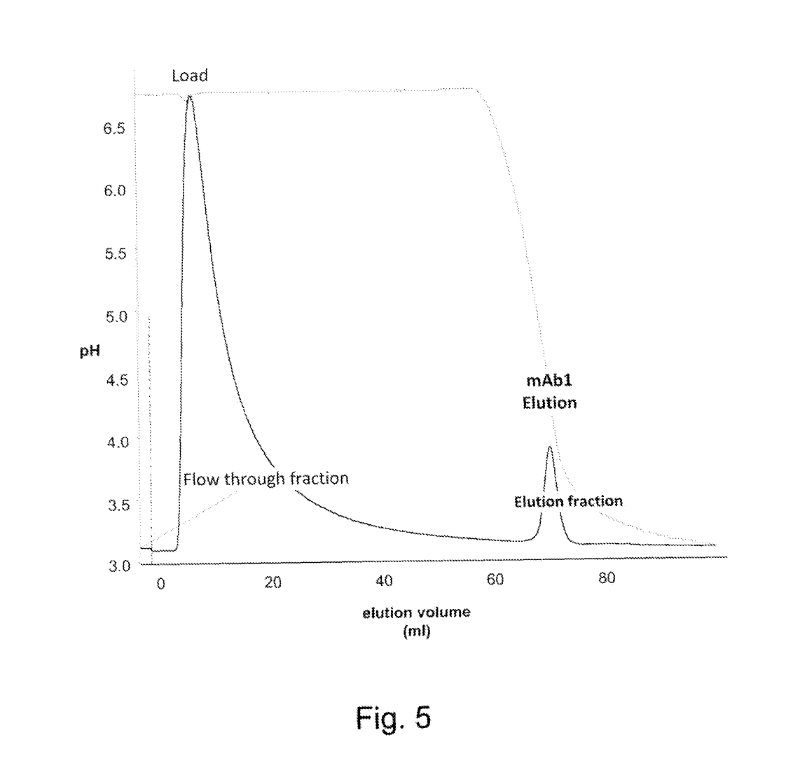

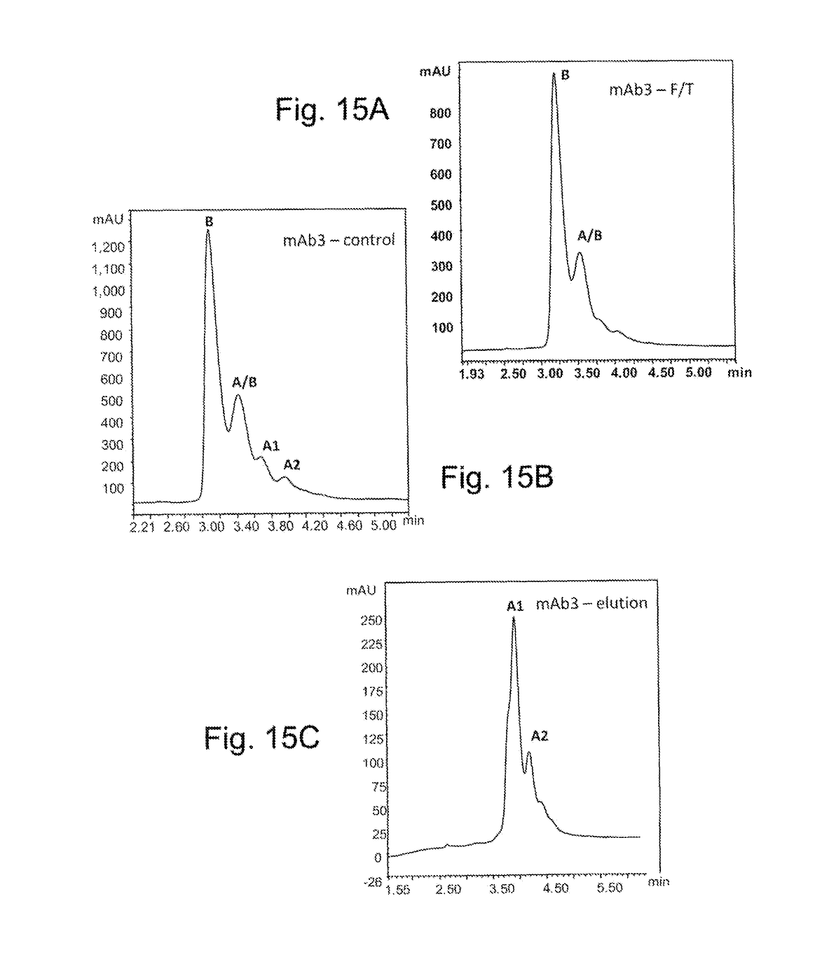

In one application, mAb3 material was loaded on a 24 mL HP-6014 affinity column and eluted while monitoring by UV detection at 214 & 280 nm (FIG. 15). A large portion of the material was not retained and was washed through with the running buffer (FIG. 15A). A smaller portion of the mAb3 material was retained and later eluted using a pH gradient from pH 7.2→2.8 (FIG. 15C). Fractions were collected across the anti-hu IgG2 affinity separation and analyzed by reversed-phase analysis (FIGS. 15A and 15C). Similar to the Protein L separation of mAb1 (FIG. 5), the IgG2-A species were retained and later eluted as the pH was lowered using the 24 mL column (FIG. 15C). No detectable amount of IgG2-B was observed in the enriched IgG2-A fraction, indicating that HP-6014 had no or weak binding to the IgG2-B species. Similar to Protein L, the anti-human IgG2 F/T fractions showed depletion of the mAb3 IgG2-A species and therefore enrichment of IgG2-B material (FIG. 15A). The IgG2-A/B isoform was observed mostly in the F/T fractions, displaying weak binding relative to IgG2-A.

Further development of this column has shown that the two IgG2-A species (A1 & A2) have significantly different binding to the anti-hu IgG2 affinity column. This was shown by injecting (below column capacity) high purity IgG2-A material (containing 60-90% A1) onto the anti-hu IgG2 affinity column and collecting the F/T and low pH elution fractions. Analysis by reversed-phase showed that IgG2-A2 in not significantly retained when high levels of IgG2-A1 are present. The above-described anti-hu IgG2 affinity column is believed to be the only technique capable of efficiently separating the IgG2-A subspecies A1 & A2.

### Example 10

### Preparative Scale Cation Exchange Chromatography—mAb7 Loaded at 12 g/L Packed Resin; 7 cm Diameter Column

Monoclonal antibody mAb7 was produced on a a preparative scale as follows. Cation exchange resin YMC BioPro S30 was packed to a 21.5 cm bed height in a 7 cm diameter column (0.83 L column volume). The column was equilibrated with 3 column volumes of 250 mM sodium chloride, 10 mM sodium acetate pH 5.2 (buffer B) followed by 4 columns of 10 mM sodium acetate pH 5.2 (buffer A). Approximately 10.2 g of IgG2 in about one column volume of buffer A was loaded onto the column. After loading, a two column volume gradient from 0% buffer B to 33% buffer B was applied to the column.

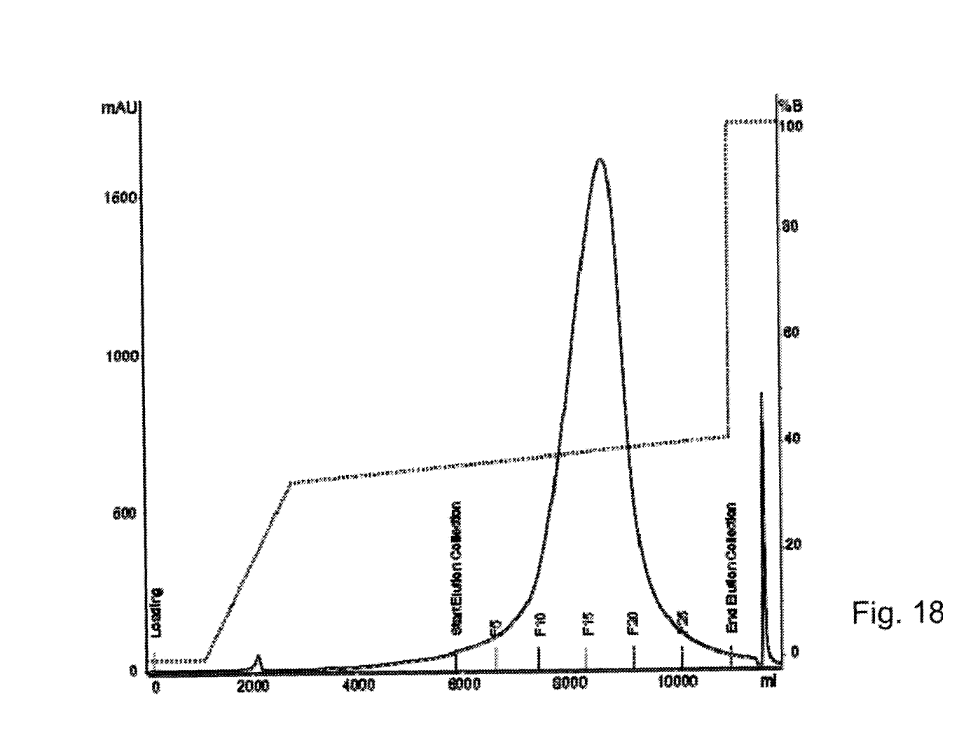

The IgG2 was eluted with a 10 column volume gradient from 33% buffer B to 41% buffer B, corresponding to a gradient slope of 2 mM sodium chloride per column volume. Fractions of 0.3 column volume were collected. FIG. 18 shows a chromatogram of the results.

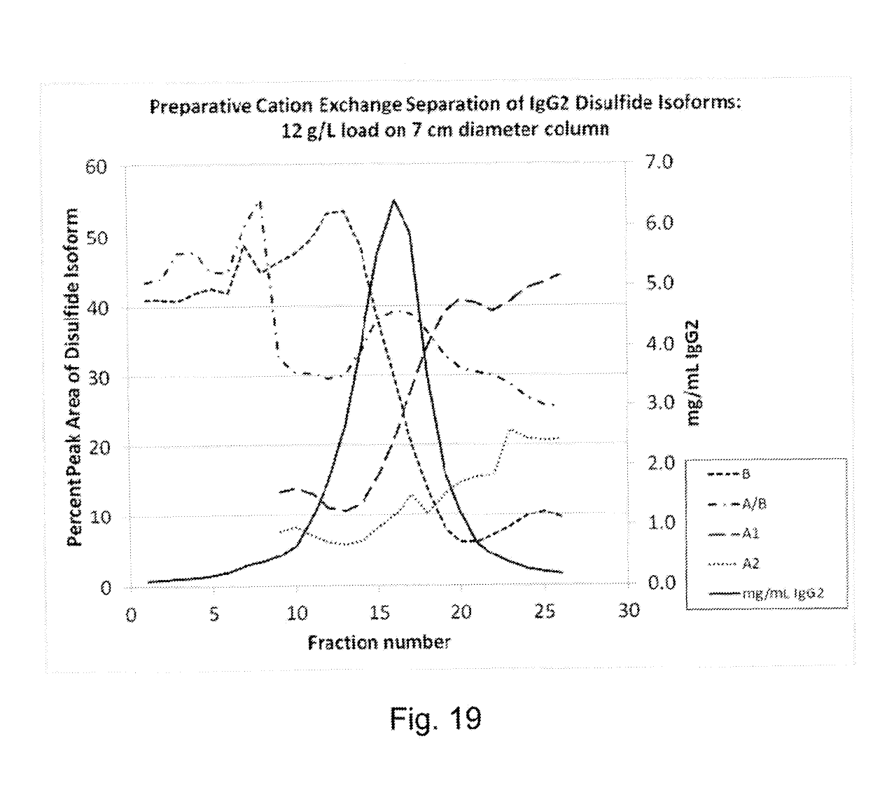

The load and samples of each fraction were assayed for protein content by measurement of absorbance at 280 nm and for disulfide isoforms by non-reduced RPHPLC. The load contained about 31% B, 36% A/B, 22% A1, and 11% A2 disulfide isoforms. FIG. 19 shows an overlay of the total IgG2 concentration and the percent peak areas for disulfide isoforms B, A/B, A1 and A2 for each fraction. Note that the β isoforms are enriched in the fractions at the front of the peak (fractions 10 to 15), and the A1 and A2 forms are enriched in the tailing fractions of the peak (fractions 18 to 26). The overall proportions of B, A/B, A1 and A2 may be adjusted in the eluted pool by selecting which fractions are pooled or by the start collect and end collect criteria for the elution. Fractions enriched for certain isoforms may be selected for additional enrichment by re-chromatography on a cation exchange column such as was used above or using further chromatography by other modes such as affinity chromatography, hydrophobic interaction chromatography or reversed phase chromatography.

### Example 11

### Preparative Scale Cation Exchange Chromatography—mAb Loaded at 2.1 g Per L of Packed Resin; 10 cm Diameter Column

A monoclonal antibody was produced on a a preparative scale as follows. Cation exchange resin YMC BioPro S30 was packed to a 27 cm bed height in a 10 cm diameter column (2.12 L column volume). The column was equilibrated with three column volumes of 400 mM sodium chloride, 10 mM sodium acetate pH 4.5 (buffer B) followed by four column volumes of 10 mM sodium acetate pH 5.2 (buffer A). Approximately 4.5 g of IgG2 in about 1 column volume of buffer A was loaded onto the column. After loading, a 2 column volume gradient from 0% buffer B to 55.5% buffer B was applied to the column.

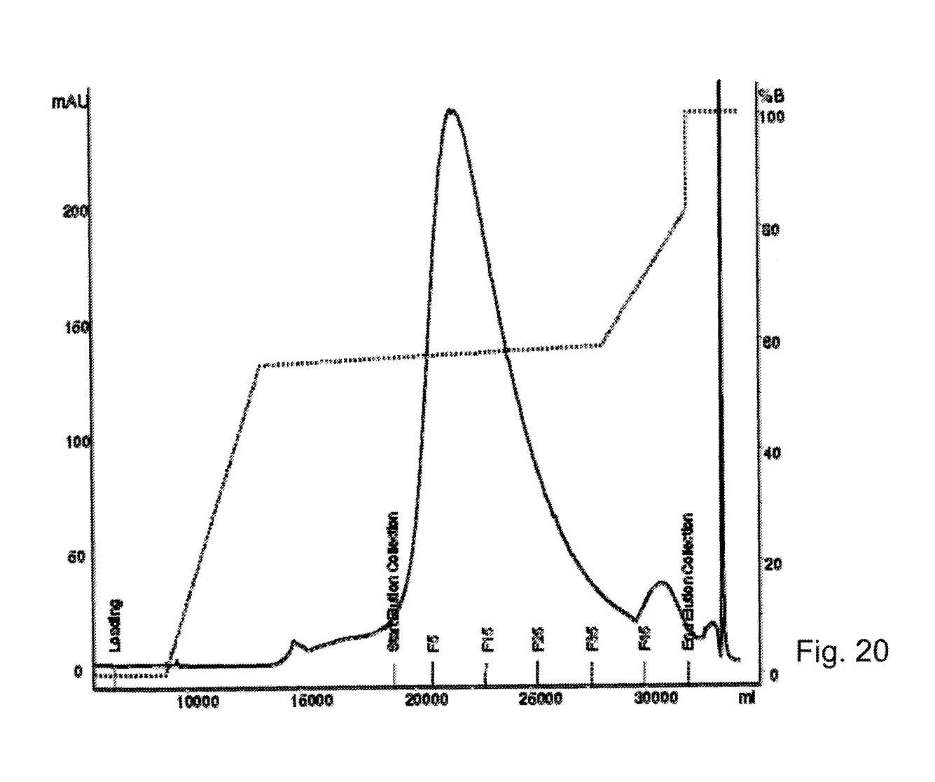

The IgG2 was eluted with a 7 column volume gradient from 55.5% buffer B to 58.7% buffer B. This corresponds to a gradient slope of 1.8 mM sodium chloride per column volume. After the main peak eluted, a short 1.8 CV gradient up to 83% buffer B was run, followed by a jump to 100% buffer B. Fractions of about 0.1 column volume were collected. FIG. 20 shows the preparative cation exchange chromatogram.

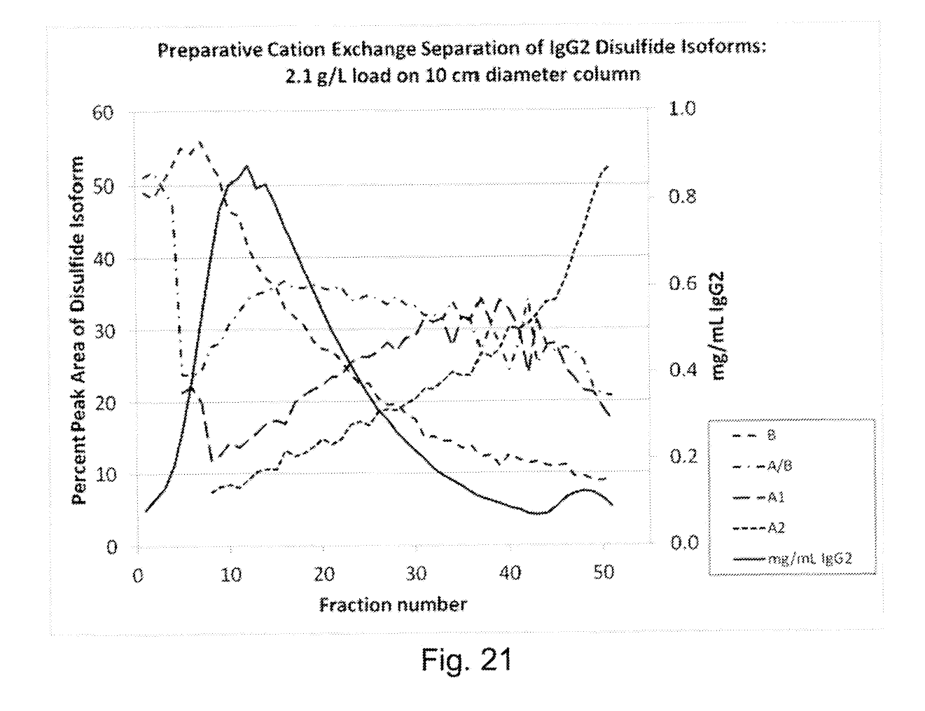

The load and samples of each fraction were assayed for protein content by measurement of absorbance at 280 nm and for disulfide isoforms by non-reduced RPHPLC. The load contained about 32% B, 33% A/B, 20% A1, and 15% A2 disulfide isoforms. FIG. 21 shows an overlay of the total IgG2 concentration and the percent peak areas for disulfide isoforms B, A/B, A1 and A2 for each fraction. It can be seen from FIG. 21 that the β isoforms are enriched in the fractions at the front of the peak (fractions 3 to 15), and the A1 and A2 forms are enriched in the tailing fractions of the peak (fractions 25 to 50). The overall proportions of B, A/B, A1 and A2 in the eluted pool can be adjusted by which fractions are pooled or by the start collect and end collect criteria for the elution. Fractions enriched for certain isoforms may be selected for additional enrichment by re-chromatography on the cation exchange column or further chromatography by other modes such as affinity chromatography, hydrophobic interaction chromatography or reversed phase chromatography.

1. A method of producing an IgG2 antibody preparation enriched for one of several IgG2 structural isoforms which differ by disulfide connectivity in the hinge region of the antibody, comprising
(A) contacting a solution containing a recombinantly-produced IgG2 antibody with a first matrix selected from the group consisting of a strong cation exchange (SCX) matrix, an anti-human IgG2 affinity matrix and a Protein L matrix, and
(B) eluting two or more first elution fractions from the first matrix,
wherein the IgG2 antibody solution to be subjected to the method elutes off the first matrix as two or more separate forms corresponding to two or more IgG2 structural isoforms, and at least one of the two or more first elution fractions is enriched for at least one of the IgG2 structural isoforms which differ by disulfide connectivity in the hinge region.

2. The method of claim 1, wherein the anti-human IgG2 affinity matrix is an HP-6014 affinity matrix, further comprising contacting at least one of the two or more first elution fractions with a second matrix selected from the group consisting of an SCX matrix, an HP-6014 affinity matrix and a Protein L matrix, and eluting two or more second elution fractions off the second matrix,
wherein at least one of the two or more second elution fractions is further enriched for at least one of the IgG2 structural isoforms.

3. The method of claim 2, wherein the first matrix is an SCX matrix and the second matrix is an HP-6014 affinity matrix.

4. The method of claim 1, wherein the first matrix is an SCX matrix and the second matrix is a Protein L matrix.

5. The method of claim 1, wherein the first matrix is an HP-6014 affinity matrix and the second matrix is an SCX matrix.

6. The method of any of claims 1-5, wherein said SCX resin is in a column and said structural isoforms are isolated in fractions of eluate from said column.

7. The method of any of claims 1-5, wherein said structural isoforms are identified by subjecting said fractions to a reversed-phase assay.

8. The method of any of claims 1-5, wherein said SCX matrix is a high-capacity YMC SCX resin.

9. The method of any of claims 1-5, wherein said structural isoforms are selected from the group consisting of IgG2-A, IgG2-A/B and IgG2-B.

10. The method of any of claims 1-5, wherein the eluting from the first or second matrix is performed using a low pH buffer.

11. The method of claim 10, wherein the buffer has a pH selected from the group consisting of between 2 and 3, between 3 and 4, between 4 and 5, between 5 and 6, and between 6 and 7.

12. The method of any of claims 1-5, wherein the elution step(s) is performed using a pH step elution.

13. The method of any of claims 1-5, wherein the elution step(s) is performed using a pH gradient elution.

14. The method of claim 14, wherein the pH gradient is varied from a pH of between 7 and 7.5 to a pH between 2.5 and 3.

15. The method of any of claims 1-5, wherein the elution step(s) is performed using a salt step elution.

16. The method of any of claims 1-5, wherein the elution step(s) is performed using a salt gradient elution.

17. The method of any of claim 16, wherein the salt gradient increases the salt concentration from about 100 mM to about 250 mM.

18. The method of any of claims 15-17, wherein the salt is NaCl.

19. The method of any of claims 1-18, wherein the IgG2 antibody preparation is enriched for at least one of the IgG2 structural isoforms to a level of at least between 20% and 50% purity of the desired IgG2 structural isoform.

20. The method of any of claims 1-19, wherein the IgG2 antibody preparation is produced on a preparative scale.

21. The method of claim 16, wherein the SCX matrix employs a salt gradient of between 1 and 2 mM salt per column volume.

22. A method of producing an IgG2 antibody preparation enriched for one of several IgG2 structural isoforms which differ by disulfide connectivity in the hinge region of the antibody, comprising
subjecting a preparation comprising an IgG2 antibody having a kappa light chain of the VκI, VκIII, or VκIV subclasses to a Protein L resin,
wherein said IgG2 antibody in said preparation elutes from said Protein L resin as two or more structural IgG2 isoforms.
